(19) United States
(12) Patent Application Publication
(10) Pub. No.: US 2015/0151142 A1
(43) Pub. Date: Jun. 4, 2015

(54) Device and Methods for Targeting of Transcranial Ultrasound Neuromodulation by Automated Transcranial Doppler Imaging

(71) Applicants: THYNC, INC., Los Gatos, CA (US); UNIVERSITY OF WASHINGTON, Seattle, WA (US)

(72) Inventors: William J. TYLER, Roanoke, VA (US); Pierre MOURAD, Seattle, WA (US); Daniel Z. WETMORE, San Francisco, CA (US)

(21) Appl. No.: 14/501,523

(22) Filed: Sep. 30, 2014

Related U.S. Application Data
(63) Continuation of application No. PCT/US13/35014, filed on Apr. 2, 2013.
(60) Provisional application No. 61/619,233, filed on Apr. 2, 2012.

(51) Int. Cl. A61N 7/00; A61B 8/00; A61B 5/0476; A61B 5/1455; A61B 5/055; A61B 5/053; A61B 6/03; A61B 8/08; A61B 5/0205

(52) U.S. Cl. CPC A61N 7/00; A61B 8/488; A61B 8/0808; A61B 8/466; A61B 8/481; A61B 8/4477; A61B 5/0476; A61B 5/0205; A61B 5/055; A61B 5/0533; A61B 6/03; A61B 5/14551; A61B 5/021

(57) ABSTRACT

Methods and systems for transcranial ultrasound neuromodulation as well as targeting such neuromodulation in the brain are disclosed. Automated transcranial Doppler imaging (aTCD) of blood flow in the brain is performed and one or more 3-dimensional maps of the neurovasculature are generated. Ultrasound energy is delivered transcranially in conjunction to induce neuromodulation. One or more brain regions for neuromodulation are targeted by using brain blood vessel landmarks identified by aTCD components. The landmarks are used for initial targeting of the neuromodulation to one or more brain regions of interest and/or for maintaining neuromodulation targeting despite user or device movements. Acoustic contrast agents may be employed to generate broadband ultrasound waves locally at the site of target cells. Transcranial ultrasound neuromodulation may be achieved by having confocal ultrasound waves differing in acoustic frequency by a frequency effective for neuromodulation interfere to generate vibrational forces in the brain that induce neuromodulation.

### CROSS-REFERENCE TO RELATED APPLICATIONS

This application is a continuation application of PCT/US2013/035014 filed on Apr. 2, 2013 (Attorney Docket No. 42043-705.601) which claims the benefit of priority of U.S. Provisional Patent Application No. 61/619,233 (Attorney Docket No. 42043-705.101) filed Apr. 2, 2012, the entire disclosures of which are incorporated herein by reference.

### FIELD OF THE INVENTION

The present invention relates to methods and systems for transcranial ultrasound neuromodulation, including methods and systems for targeting transcranial ultrasound neuromodulation in the brain.

### BACKGROUND OF THE INVENTION

Ultrasound (hereinafter “US”) has been used for many medical applications, and is generally known as cyclic sound pressure with a frequency greater than the upper limit of human hearing. An important benefit of ultrasound therapy is its non-invasive nature. US waveforms can be defined by their acoustic frequency, intensity, waveform duration, and other parameters that vary the timecourse of acoustic waves in a target tissue.

When used for imaging, ultrasonic transducers are provided with several transducer elements arranged in an array and driven by different voltages. By controlling the phase and amplitude of the applied voltages, ultrasonic waves combine to produce a net ultrasonic wave that travels along a desired beam direction and is focused at a selected point along the beam. By controlling the phase and the amplitude of the applied voltages, the focal point of the beam can be moved in a plane to scan the subject. Many such ultrasonic imaging systems are well known in the art, including those that employ arrays of piezoelectric transducers or arrays of capacitive micromachined transducers (hereinafter “CMUTs”).

Doppler ultrasound has been in use in medicine for many years. Doppler ultrasound techniques measure the frequency shift (the “Doppler Effect”) of reflected sound, which indicates the velocity of the reflecting material. Long-standing applications of Doppler ultrasound include monitoring of the fetal heart rate during labor and delivery and evaluating blood flow in the carotid artery. The use of Doppler ultrasound has expanded greatly in the past two decades, and Doppler ultrasound is now used in many medical specialties, including cardiology, neurology, radiology, obstetrics, pediatrics, and surgery.

Transcranial Doppler (hereinafter “TCD”) ultrasonography provides an easy-to-use, non-invasive, non-radioactive, and relatively inexpensive method to assess locate, track, and evaluate hemodynamics in blood vessels in the brain. Velocities from the cerebral arteries, the internal carotids, the basilar, and the vertebral arteries can be sampled by altering the transducer location and angle, and the instrument's depth setting. The most common windows in the cranium are located in the orbit (of the eye), and in the temporal and suboccipital regions.

One drawback of measuring physiological parameters using a standard TCD probe is that identifying a desired target site using a TCD probe is challenging and generally requires a trained, experienced sonographer to find and (acoustically) illuminate a desired target site, such as the middle cerebral artery (MCA). When longer term monitoring of physiological parameters using a TCD probe is required, a cumbersome and in many instances uncomfortable headset having the TCD probe mounted can be mounted on the subject's head to stabilize the transducer position and reduce the effects of patient movement and other disturbances on the position of the probe. The sonographer may be required to monitor acoustic readings and reposition the transducer intermittently to maintain the focus on the desired data acquisition area.

Several systems for extended automated TCD (hereinafter “aTCD”) monitoring have been proposed that address the methodological challenges of standard TCD monitoring as described in the preceding paragraph. These methods are particularly advantageous, because they generally enable unattended TCD imaging. By using an aTCD system, imaging of blood vasculature can proceed with intermittent oversight by a technician or without a technician overseeing the TCD targeting. Previous patents and publications have described aTCD systems. U.S. Pat. No. 6,682,483 discloses methods and devices that provide three dimensional imaging of blood flow using long-term, unattended Doppler ultrasound techniques. U.S. Pat. No. 7,547,283 discloses a head-set arrangement wherein a transducer array and array electronics are permanently mounted on a structure facilitating communication to and from a controller component.

Recent research and disclosures have described the use of transcranial ultrasound neuromodulation to activate, inhibit, or modulate neuronal activity. See e.g., Bystritsky et al., 2011; Tufail et al., 2010; Tufail et al., 2011; Tyler et al., 2008; Yang et al., 2011; Yoo et al., 2011; Zaghi et al., 2010, the full disclosures of which are incorporated herein by reference. Also see e.g., U.S. Pat. Nos. 7,283,861 and U.S. Publication Nos. 2007/0299370 and 2011/0092800, entitled “Methods for Modifying Currents in Neuronal Circuits” by Alexander Bystritsky; U.S. Patent Application No. 2008/0045882, entitled “Biological Cell Acoustic Enhancement and Stimulation” by Finsterwald; U.S. patent applications Ser. No. 13/003,853 (published as U.S. Publication No. 2011/0178441), entitled “Methods and Devices for Modulating Cellular Activity Using Ultrasound”; PCT Application No. PCT/US2010/055527 (published as PCT Publication No: WO/2011/057028), entitled “Devices and Methods for Modulating Brain Activity”; U.S. Application No. 61/550,334, entitled “Improvement of Direct Communication.” Transcranial ultrasound neuromodulation is an advantageous form of brain stimulation due to its non-invasiveness, safety, focusing characteristics, and the capacity to vary transcranial ultrasound neuromodulation waveform protocols for specificity of neuromodulation.

To affect brain function transcranial ultrasound neuromodulation requires appropriate ultrasound waveform parameters, including acoustic frequencies generally less than about 10 MHz, spatial-peak temporal-average intensity generally less than about 10 W/cm², and appropriate pulsing and other waveform characteristics to ensure that heating of a targeted brain region does not exceed about 2 degrees Celsius for more than about 5 seconds. Transcranial ultrasound neuromodulation induces neuromodulation primarily through vibrational or mechanical mechanisms. Noninvasive and nondestructive transcranial ultrasound neuromodulation is in contrast to other transcranial ultrasound based techniques that use a combination of parameters to disrupt, damage, destroy, or otherwise affect neuronal cell populations so that they do not function properly and/or cause heating to damage or ablate tissue.

Although prior systems and methods for transcranial ultrasound neuromodulation deliver ultrasound at acoustic frequencies for inducing neuromodulation in the brain, the absorption of ultrasound by bone can be highly dependent on the acoustic frequency with more absorption at frequencies greater than about 1 MHz, and the treatment can be less than ideal in at least some instances. Although ultrasound below about 0.7 MHz can be transmitted more effectively through bone than ultrasound above 1 MHz, prior transcranial ultrasound neuromodulation achieved by delivering ultrasound with dominant acoustic frequencies<0.7 MHz, can provide less than ideal results in at least some instances. For example, the spatial resolution of the focused beam can be somewhat larger than would be ideal, and may result in less specific targeting of the target site than would be ideal in a least some instances.

Although prior ultrasound imaging systems may use higher acoustic frequencies greater than about 1 MHz, such prior systems are less than ideally suited for stimulating neurons and neuronal tissue. The neuronal tissue can be less sensitive to the ultrasound waves than would be ideal, and the high frequencies over 1 MHz can be less effective in stimulating the neuronal tissue than would be ideal.

Prior systems for transcranial ultrasound neuromodulation often lack capacity to accurately and precisely target brain regions, and such systems can be less than ideally suited for treatment in at least some instances. For example, these systems may be inaccurate in directing ultrasound energy to a desired brain region or may have difficulty directing ultrasound energy to a target site to have a desired spot size or to an exact desired location.

The prior methods and systems inadequately target transcranial ultrasound neuromodulation. Improved systems for targeting transcranial ultrasound neuromodulation based on neuroanatomy itself or fiduciary landmarks defined by neurovasculature including large blood vessels in the brain and smaller vessels in the vasculature network would be advantageous. A system that achieves both transcranial ultrasound neuromodulation and aTCD functions would be advantageous for initial targeting of transcranial ultrasound neuromodulation based on neuroanatomical targets of interest as well as adjusting targeting of transcranial ultrasound neuromodulation based on movements of the user, brain, or transcranial ultrasound neuromodulation system. Ideally, such a system would be comfortably worn by a user, and automatically adjust targeting of the transcranial ultrasound.

### SUMMARY OF THE INVENTION

Embodiments of the present invention provide improved methods and systems of ultrasound delivery in order to stimulate neuronal tissue, and overcome at least some of the deficiencies of the prior systems and methods.

In many embodiments ultrasound waves are delivered transcranially with a plurality of high ultrasound frequencies in order to localize the focused beam to a target site of decreased size, and the plurality of high ultrasound frequencies interfere at the target site to generate one or more low frequencies in order to stimulate the neuronal tissue with the one or more low frequencies. In many embodiments, the neuronal tissue is more responsive to the one or more low frequencies than to the plurality of high ultrasound frequencies, and increased amounts of stimulation can be provided with increased spatial resolution and decreased amounts of energy. The plurality of high ultrasound frequencies can be focused to the target site confocally. In many embodiments, the overlap of the two or more high frequency ultrasound beams at the target site increases interaction of the beams at the treatment site and decreases interaction of the treatment beams away from the target site where the beams do not overlap substantially, in order to provide more accurate modulation of neuronal activity at the target site.

In many embodiments, automated transcranial Doppler imaging (aTCD) of blood flow in the brain is used to generate one or more 3-dimensional maps of blood vessels in the brain, in conjunction with delivery of ultrasound energy transcranially to induce neuromodulation. Mapping the vessels of the brain in conjunction with delivering ultrasound energy allows the ultrasound energy to be delivered more accurately to desired target sites within the brain in order to induce a desired form of neuromodulation. Improved targeting can reduce the risks of unwanted neuromodulation or thermal or mechanical damage while providing improved efficacy. In many embodiments, aTCD and transcranial ultrasound neuromodulation are combined in order to target one or more brain regions for neuromodulation based on brain blood vessel landmarks identified by aTCD components. In many embodiments, the brain blood vessel landmarks are used for initial targeting of transcranial ultrasound neuromodulation to one or more brain regions, and the brain blood vessel landmarks can be used to maintain alignment of transcranial ultrasound neuromodulation on the targeted region in response to movement, such as movement of the user or movement of the components of the device. Acoustic contrast agents may be employed to generate broadband ultrasound waves locally at the site of cells to be modulated.

In many embodiments, transcranial ultrasound neuromodulation is achieved with a vibroacoustic stimulation method in which confocal ultrasound waves differing in acoustic frequency by a frequency effective for transcranial ultrasound neuromodulation interfere to generate vibrational forces in the brain that induce neuromodulation. The frequencies of the confocal ultrasound waves may be selected to have one or more of frequency, intensity, pulse length, and waveform characteristics to provide improved transmission of ultrasound energy through a subject's skull and to the target in the brain without causing significant thermal or mechanical damage. The frequencies of the confocal ultrasound waves may be selected so that one or more of frequency, intensity, pulse length, and waveform characteristics of the frequency difference between the confocal ultrasound waves can induce neuromodulation without causing significant thermal or mechanical damage.

In a first aspect, an apparatus to treat a subject with ultrasound energy is provided. The apparatus comprises two or more ultrasound transducers and circuitry coupled to the two or more transducers. The ultrasound transducers direct ultrasound energy transcranially to a neuronal target site of the subject. The circuitry drives the transducers with two or more ultrasound frequencies in order to treat the subject with one or more ultrasound frequencies that are less than the two or more ultrasound frequencies. In some embodiments, the transducers and the circuitry may be configured (i) to map the subject transcranially and to track a tissue site of the subject with at least one of the two or more ultrasound frequencies and (ii) to treat the target site with the two or more ultrasound frequencies. The two or more ultrasound frequencies may comprise frequencies within a range from about 1 MHz to about 15 MHz. The one or more frequencies less than the ultrasound frequency may comprise frequencies less than about 1 MHz. In other embodiments, the transducers and the circuitry may be configured to map the subject transcranially and to track a tissue site of the subject with a tracking ultrasound frequency different from the two or more ultrasound frequencies for treating the subject.

The circuitry and transducers may be configured to adjust an angle of the target site relative to the transducers in response to movement of the tissue relative to the transducers. The circuitry and transducers may also be configured to adjust the angle and a depth of the target site relative to the transducers in response to movement of the tissue relative to an angle and a depth of the transducers. This tissue may be untreated tissue that may be concurrently tracked or may be tissue that is currently being targeted for treatment.

The transducers may comprise confocal transducers to direct the ultrasound to the target site and the circuitry may be configured to drive the confocal transducers with the two or more ultrasound frequencies. The two or more ultrasound frequencies may comprise a first ultrasound frequency and a second ultrasound frequency. The one or more frequencies may comprise a difference between the first ultrasound frequency and the second ultrasound frequency in order to vibrate the target site with an ultrasound frequency based on the difference between the first frequency and the second frequency. This vibration will generally be effective to induce neuromodulation.

In some embodiments, the ultrasound transducers and the circuitry are configured to map a brain blood vessels of the subject with a first transcranial ultrasound configuration (e.g., a first vibroacoustography stimulation configuration) and to treat the target site with second transcranial ultrasound configuration (e.g., a second vibroacoustography stimulation configuration).

In another aspect, a method of treating a subject with ultrasound energy is provided. Ultrasound energy comprising two or more ultrasound frequencies is directed to a target site. The two or more frequencies induce vibration of the target site with one or more ultrasound frequencies less than the two or more ultrasound frequencies to modulate neuronal activity at the target site. The subject's brain blood vessels may also be mapped transcranially and a tissue site may also be tracked with at least one of the two or more ultrasound frequencies. In some embodiments, the frequency applied to track the tissue site is different from the frequencies applied to treat the target site. An angle of ultrasound energy direction to the target site may be adjusted in response to movement of the tracked tissue site. The angle and a depth of the target site may also be adjusted in response to movement of the tracked tissue site. The tracked tissue site may comprise an untreated tissue site or may be the target site itself. The two or more ultrasound frequencies may comprise frequencies within a range from about 1 MHz to about 15 MHz and the one or more frequencies less than the ultrasound frequency comprise frequencies may be less than about 1 MHz.

Generally, the two or more ultrasound frequencies comprise a first ultrasound frequency and a second ultrasound frequency. The one or more ultrasound frequencies less than the two or more ultrasound frequencies may comprise a difference between the first ultrasound frequency and the second ultrasound frequency. The direction of ultrasound energy may vibrate the target site with an ultrasound frequency based on the difference between the first ultrasound frequency and the second ultrasound frequency.

In some embodiments, ultrasound energy is directed to map brain blood vessels of the subject with a first transcranial ultrasound configuration (e.g., a first vibroacoustography stimulation configuration) and the target site is treated with a second transcranial ultrasound configuration (e.g., a second vibroacoustography stimulation).

In a further aspect, an apparatus for treating a subject with ultrasound energy is provided. The apparatus comprises a processor configured to implement any one of the methods described herein.

In yet another aspect of the invention, a system for transcranial ultrasound neuromodulation is provided. The system uses Doppler ultrasound imaging for targeting one or more brain regions. The transcranial Doppler imaging system may be an automated transcranial Doppler (aTCD) imaging system.

The aTCD will generally be used to generate a three-dimensional map of brain blood vessels. A first three-dimensional map of brain blood vessels generated may be stored in machine-readable format. One or more subsequent brain blood vessel maps generated by aTCD may image a subset of one or more brain blood vessels mapped in the detailed three-dimensional map. The one or more subsequent brain blood vessel maps generated by aTCD may be used to target transcranial ultrasound neuromodulation to one or more brain regions. The detailed three-dimensional map of brain blood vessels generated by aTCD may be repeated intermittently.

The three-dimensional map of brain blood vessels generated by aTCD can serve as a fiduciary landmark for targeting transcranial ultrasound neuromodulation. This map may be created before a transcranial ultrasound neuromodulation session. This map may also be updated during a transcranial ultrasound neuromodulation session, for example updated more than one per hour, more than once per minute, or more than once per second. Updates may be made continuously. A change in the relative position of the one or more targeted brain regions and one or more transcranial ultrasound neuromodulation transducers during a transcranial ultrasound neuromodulation session can be determined by comparing two or more aTCD images. The position or orientation of one or more transcranial ultrasound neuromodulation transducers may be automatically changed based on the relative movement detected in order to maintain targeting of one or more brain regions. The focusing characteristics of one or more transcranial ultrasound neuromodulation transducers may be automatically changed based on the relative movement detected in order to maintain targeting of one or more brain regions. The accuracy of targeting for transcranial ultrasound neuromodulation may be less than 1 cm³ or less than 1 mm³.

The three-dimensional map of brain blood vessels may be stored in machine readable format. The machine readable map of brain blood vessels may be stored in many places, for example, in one or more components of the device wearably attached to the subject or remotely on a server.

The delivered ultrasound energy may have various properties to achieve a desired neuromodulation. The spatial-peak, temporal-average intensity in brain tissue for transcranial ultrasound neuromodulation may be chosen from a range of about 0.0001 mW/cm² to about 1 W/cm². The heating of brain tissue at the target location may be no more than about 2 degrees Celsius for no more than about 5 seconds. The acoustic frequency for transcranial ultrasound neuromodulation may be in a range between about 100 kHz and about 1 MHz, and this acoustic frequency may be modulated during the transcranial ultrasound neuromodulation protocol. The acoustic frequency for aTCD may be in a range between about 0.5 MHz and about 15 MHz, and this acoustic frequency may be modulated during aTCD imaging.

Transcranial ultrasound neuromodulation can be implemented in many ways. Two confocal ultrasound transducers differing in dominant acoustic frequency by an acoustic frequency appropriate for transcranial ultrasound neuromodulation may be targeted at a site of tissue to be modulated by transcranial ultrasound neuromodulation. A transcranial ultrasound neuromodulation protocol may be targeted to multiple brain regions with one or more ultrasound transducers. Multiple transcranial ultrasound neuromodulation protocols differing in one or more of spatial-peak, temporal-average intensity, acoustic frequency, pulse length, pulse repetition frequency, and number of pulses may be delivered concurrently or in series to one or more brain regions from one or more ultrasound transducers. The transcranial ultrasound neuromodulation transducers may target one or more brain regions chosen from the list of: primary sensory cortex, primary and secondary motor cortex, association cortex (including areas involved in emotion, executive control, language, and memory), other region of cerebral cortex, the limbic system (including the amygdala), hippocampus, parahippocampal formation, entorhinal cortex, subiculum, thalamus, hypothalamus, white matter tracts, brainstem nuclei, cerebellum, neuromodulatory system, or other brain region. The transcranial ultrasound neuromodulation stimulation may be perceived subjectively by the recipient as a sensory perception, movement, concept, instruction, other symbolic communication, or modifies the recipient's cognitive, emotional, physiological, attentional, or other cognitive state. The system may include one or more components for measuring brain activity that takes the form of one or a plurality of: electroencephalography (EEG), magnetoencephalography (MEG), functional magnetic resonance imaging (fMRI), functional near-infrared spectroscopy (fNIRS), positron emission tomography (PET), single-photon emission computed tomography (SPECT), computed tomography (CT), or other techniques for measuring brain activity. The brain activity may be measured by detecting changes in hemodynamics with aTCD or fTPI. The system includes one or more components for a physiological measurement of the body that takes the form of one or a plurality of: electromyogram (EMG), galvanic skin response (GSR), heart rate, blood pressure, respiration rate, pulse oximetry, pupil dilation, eye movement, gaze direction, or other physiological measurement. The transcranial ultrasound neuromodulation protocol can include modulation of one or more stimulus parameters chosen from spatial-peak, temporal-average intensity, acoustic frequency, pulse repetition frequency, number of pulses, and pulse length. Broadband ultrasound may be generated at the site of tissue to be modulated through the use of an acoustic contrast agent.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Embodiments have other advantages and features which will be more readily apparent from the following detailed description of the invention and the appended claims, when taken in conjunction with the accompanying drawings, in which:

FIG. 1A shows an apparatus to target and deliver transcranial ultrasound neuromodulation in accordance with many embodiments;

FIG. 1B shows the apparatus of FIG. 1A having a subject interface in the form of an annular array in accordance with some embodiments;

FIG. 1C shows the apparatus of FIG. 1A having a subject interface in the form of a patch placed on the subject's head in accordance with some embodiments;

FIG. 2A shows a method for transcranial ultrasound neuromodulation delivery in accordance with many embodiments;

FIG. 2B shows a method to adjust targeting of transcranial ultrasound neuromodulation based on aTCD in accordance with many embodiments;

FIG. 3 shows a transcranial ultrasound neuromodulation waveform and a pulsed ultrasound protocol, in accordance with many embodiments;

FIG. 4 shows a transcranial ultrasound neuromodulation waveform, and a continuous wave ultrasound protocol, in accordance with many embodiments;

FIG. 5 shows transcranial ultrasound neuromodulation waveform repetition, in accordance with many embodiments;

FIGS. 6A and 6B show a schematic showing the use of a transducer array for aTCD in scanning mode (FIG. 6A) and focused data acquisition mode (FIG. 6B), in accordance with many embodiments;

FIGS. 7A and 7B show a vibroacoustography stimulation system and associated signals, in accordance with many embodiments;

FIG. 8 shows a schematic of mouse movements assessed by vibroacoustic stimulation in an exemplary embodiment;

FIG. 9 shows movements elicited by stimulation at various locations transcranially in a mouse in an exemplary embodiment; and

FIG. 10 shows movements elicited by stimulation at various locations transcranially in a mouse in an exemplary embodiment.

### DETAILED DESCRIPTION

The embodiments as described herein can be used in one or more of many ways to beneficially treat neurons of a subject with ultrasound energy. The neurons may comprise neurons of a brain of a subject, and the ultrasound can be delivered transcranially, for example. The embodiments as described herein can be beneficially combined to provide method and apparatus to treat modulate neuronal activity of a target site of the subject, and the target site may comprise a neuronal site. In many embodiments, high frequency components of ultrasound are combined to provide low frequency vibrational energy to modulate the target site. Alternatively or in combination, mapping of blood vessels can be used to identify target locations by spatial reference to blood vessels.

In many embodiments, transcranial ultrasound neuromodulation protocols are used to direct ultrasound energy to a targeted region of the brain of a human or animal based on identified locations in the brain of the patient. The ultrasound energy directed to the targeted region of the brain can modulate neuronal activity, and can activate or inhibit the neuronal activity, for example. In many embodiments, the ultrasound energy modulates neuronal activity through mechanical effects when delivered with an appropriate ultrasound waveform, for example an appropriate ultrasound waveform as described herein. The effective targeting of transcranial ultrasound neuromodulation as described herein can provide beneficial results and be used to identify and target one or more regions of the brain.

Automated transcranial Doppler (aTCD) as described herein can be used identify blood vessels of the brain to identify a target region of the brain based on the location of the blood vessel. In many embodiments, the 3-dimensional map of blood vessels defined by aTCD provides a fiduciary or landmark for locating neuroanatomical regions or structures on the basis of the relative location of the neuroanatomical regions relative to a defined 3-dimensional map of blood vessels. Embodiments include one or more components for aTCD imaging that is a non-invasive, continuous, and unattended system that achieves 3-dimensional blood vessel tracking via transcranial ultrasound Doppler.

Systems and methods described herein combine aTCD and transcranial ultrasound neuromodulation for improved targeting of transcranial ultrasound neuromodulation based on mapping the brain region targeted by ultrasound neuromodulation in relation to brain blood vessels located with aTCD.

In many embodiments, one or more components of transcranial ultrasound neuromodulation systems and methods are combined with one or more components of transcranial Doppler ultrasound systems and methods, in order to accurately direct the ultrasound energy to a targeted region. The transcranial Doppler ultrasound can be used to identify portions of one or more blood vessels in order to accurately direct the treatment to the targeted region.

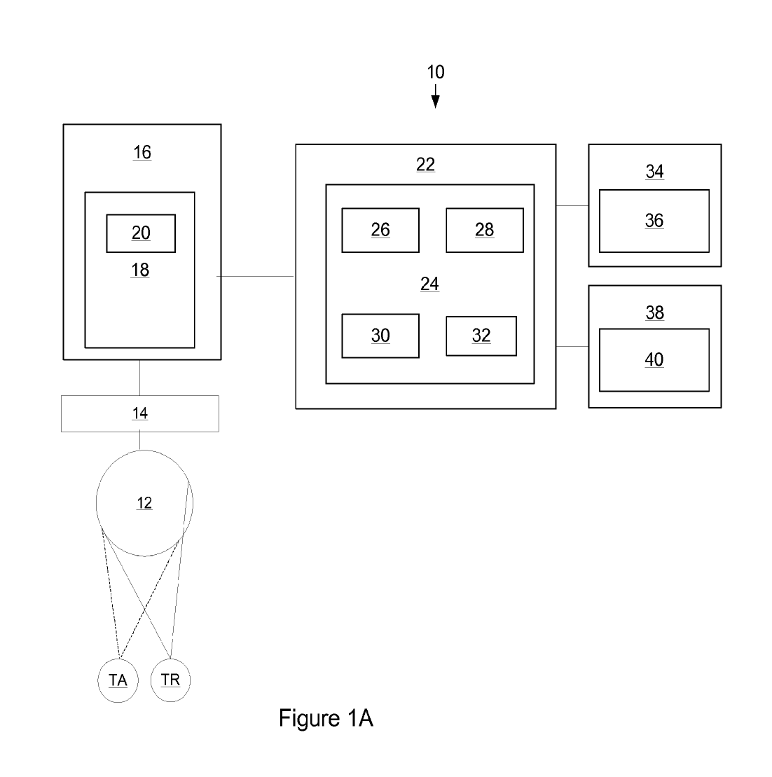

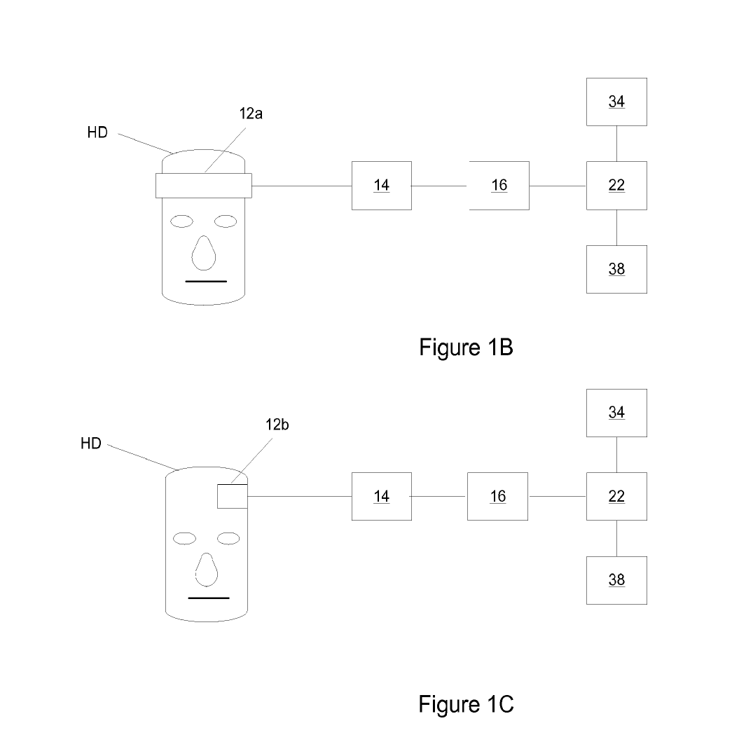

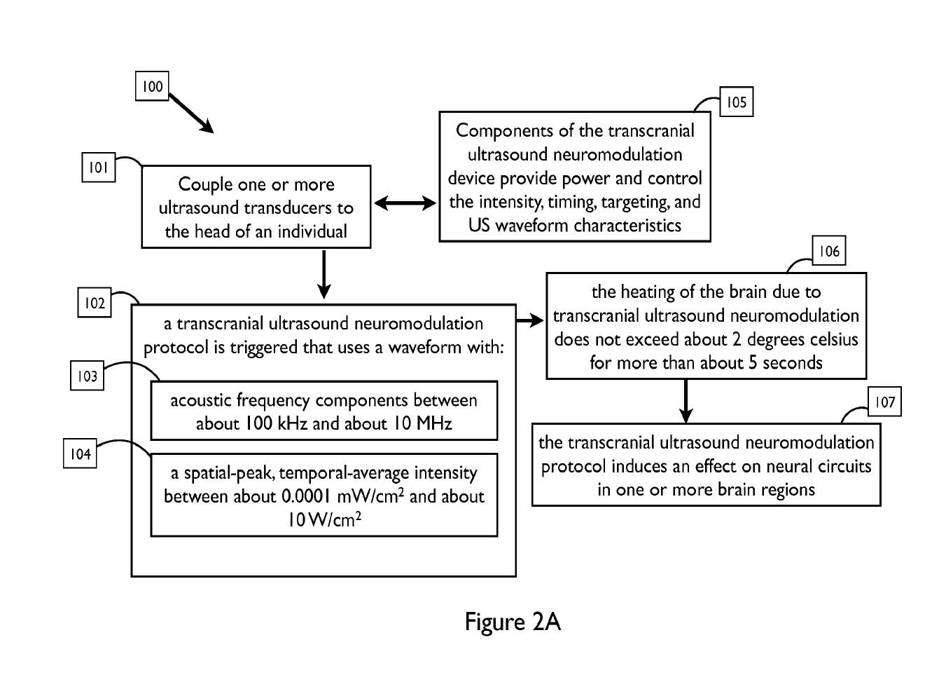

FIG. 1A shows an apparatus 10 to target and deliver transcranial ultrasound neuromodulation in accordance with many embodiments. The apparatus 10 comprises an ultrasound source 12, circuitry 14, and a controller 16. The circuitry 14 can drive the ultrasound source 12 in one or more desired frequencies in accordance with instructions from the controller 16. The ultrasound source 12 may comprise a plurality of ultrasound transducers, for example an array of ultrasound transducers. The ultrasound source 12 may at the same time deliver and receive energy to track a tracking area TR and treat a target region TA in the brain. The controller 16 comprises a processer 18 having a computer readable medium 20. The computer readable memory 20 may comprise instructions for controlling the ultrasound source 12.

In many embodiments, the ultrasound source 12 may comprises a plurality of arrays of ultrasound transducers comprising a first ultrasound array and a second ultrasound array. The first ultrasound array can be configured to provide a first focused ultrasound beam comprising a first one or more high frequencies, and second ultrasound array can be configured to provide a second focused ultrasound beam comprising a second one or more high frequencies. The first beam can be focused to a common target location with the second beam such that the first beam and the second beam comprise a confocal arrangement. The first array and the second array can be configured in one or more of many ways to provide the confocal arrangement, and may comprise a first annular array and a second annular array, for example. Alternatively or in combination, the first array and the second array may comprise grids, linear arrays, portions of annuli or other pattern suitable for providing a confocal arrangement as described herein, for example. The first array and the second array can be arranged such that the first array does not overlap substantially with the second array, and such that the first beam and the second beam do not overlap substantially away from the target site, in order to decrease interaction of the beams with tissue away from the target site. The first beam and the second beam may be transmitted through the cranium with a substantially non-overlapping configuration, for example. As the beams converge toward the target site, the first beam overlaps at least partially with the second beam in order to provide increased interaction of the beams at the target site. In many embodiments, the first array and the second array are arranged such that the beams substantially overlap only within a target region of the brain, for example. The substantial overlap may comprise an overlap of at least 50% of the area of the full width half maximum of each beam.

The apparatus 10 comprises a processor system 22. The processor system 22 is coupled with the control system 16. The processor 22 comprises a computer readable memory 24 having instructions of one or more computer programs embodied thereon. The computer readable memory 24 may comprise various instructions to perform various tasks described herein. The computer readable memory 24 may comprise instructions 26, 28, 30, 32, and optionally more or fewer instructions. The instructions 26 may comprise one or more instructions to implement method 100 described herein. The instructions 28 may comprise one or more instructions to implement one or more steps of the methods as described herein, for example method 200 described herein. The instructions 30 may comprise one or more instructions to generate a transcranial ultrasound neuromodulation waveform using a pulsed ultrasound protocol as described herein. The instructions 32 may comprise one or more instructions to generate transcranial ultrasound neuromodulation waveforms using a continuous wave ultrasound protocol.

For each method as described herein, a person of ordinary skill in the art will recognize many adaptations and variations based on the teachings described herein. For example, steps may be added or removed. Each of the steps may comprise sub-steps, and the steps may be performed in different order.

The processor system 22 is coupled to a user interface 34. The user interface 34 may comprise a display 36 such as a touch screen display. The user interface 34 may comprise a handheld device such as a commercially available iPhone, Android operating system device, such as, a Samsung Galaxy S3 or other known handheld device such as an iPad, tablet computer, or the like. The user interface 34 can be coupled with a processor system 22 with communication methods and circuitry. The communication may comprise one or more of many known communication techniques such as WiFi, Bluetooth, cellular data connection, and the like. The processor system 22 is configured to communicate with a measurement apparatus 38. The measurement apparatus 38 comprises patient measurement data storage 40 that can be stored on a computer readable memory. The processor system 22 is in communication with the measurement apparatus 38 with communication that may comprise known communication as described herein. The processor system 22 is configured to communicate with the controller 16 to transmit the signals for use with the ultrasound source 12 in for implementation with one or more components of control system 16 as described herein. The apparatus 10 allows ultrasound stimulation adjustments in variables such as carrier frequency and/or neuromodulation frequency, pulse duration, and pulse pattern, as well as the direction of the energy emission, intensity, frequency, phase/intensity relationships to targeting and accomplishing up-regulation and/or down-regulation, dynamic sweeps, and position. The user can input these parameters with the user interface 34, for example.

The ultrasound source 12 may be in many forms as described herein. FIG. 1B shows an example of the ultrasound source 12 in the form of a 3-dimensional array 12a arranged as an annular ring to be placed around a subject's head HD as in a head band. As shown in FIG. 1B, the annular array 12a is coupled to circuitry 14 for driving the array 12a, and the circuitry 14 is coupled to the processor system 22 which is coupled with the user interface 34 and the measurement apparatus 38 described herein. FIG. 1C shows an example of the ultrasound source 12 in the form of a 2-dimensional array 12b arranged as a patch to be placed on the surface of a subject's head HD. It will be appreciated that the ultrasound source 12 may instead comprise other various forms for interfacing with the subject as well. As shown in FIG. 1C, the 2-dimensional array 12b is coupled to circuitry 14 for driving the array 12b, and the circuitry 14 is coupled to the processor system 22 which is coupled with the user interface 34 and the measurement apparatus 38 described herein.

The processor, controller and control electronics and circuitry as described herein can include one or more of many suitable components, such as one or more processor, one or more field-programmable gate array (FPGA), and one or more memory storage devices. In many embodiments, the control electronics controls the control panel of the graphic user interface (hereinafter “GUI”) to provide for pre-procedure planning according to user specified treatment parameters as well as to provide the subject control over the ultrasound parameters.

The system 100 can be used to implement one or more steps of the methods as described herein, and the methods can be combined. In many embodiments, the processor and circuitry are configured with instructions of a computer readable program to perform one or more of the steps of the methods as described herein.

Transcranial Ultrasound Neuromodulation

Transcranial ultrasound neuromodulation is a technique for modulating brain circuit activity via patterned, local vibration of brain tissue using ultrasound (US) having an acoustic frequency greater than about 100 kHz and less than about 10 MHz. In many embodiments, ultrasound energy in a transcranial ultrasound neuromodulation waveform provides ultrasound energy within a range of acoustic frequencies. In many embodiments, the transcranial ultrasound neuromodulation transmits mechanical energy through the skull to the targeted region in the brain without causing significant thermal or mechanical damage and induces neuromodulation. In many embodiments, transcranial ultrasound neuromodulation employs low intensity ultrasound such that the spatial-peak, temporal-average intensity (Iₛₚₜₐ) of the transcranial ultrasound neuromodulation protocol provides less than about 10 W/cm² (preferably less than about 1 W/cm²) in the targeted brain tissue. The acoustic intensity measure Iₛₚₜₐ can be calculated according to established techniques that relate to the ultrasound acoustic pressure and other transcranial ultrasound neuromodulation protocol characteristics such as the temporal average power during the transcranial ultrasound neuromodulation waveform duration. US may be delivered as short-lived continuous waves less than about 5 seconds, in a pulsed manner, or in the form of an ultrasound waveform of arbitrary complexity during transcranial ultrasound neuromodulation protocols such that diverse patterns of neuromodulation can be delivered. For modulating the activity of brain circuits through localized tissue vibration, transcranial ultrasound neuromodulation protocols may utilize US waveforms of any type known in the art. These include amplitude modulated waveforms, tone-bursts, pulsed waveforms, continuous waveforms, and other waveform patterns as described herein, for example.

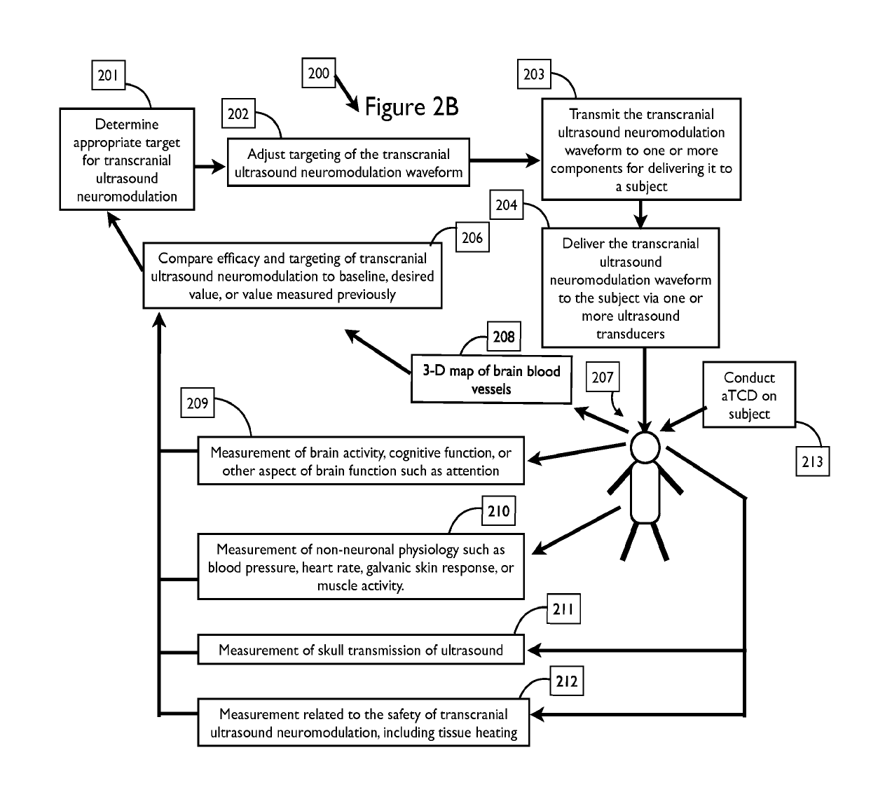

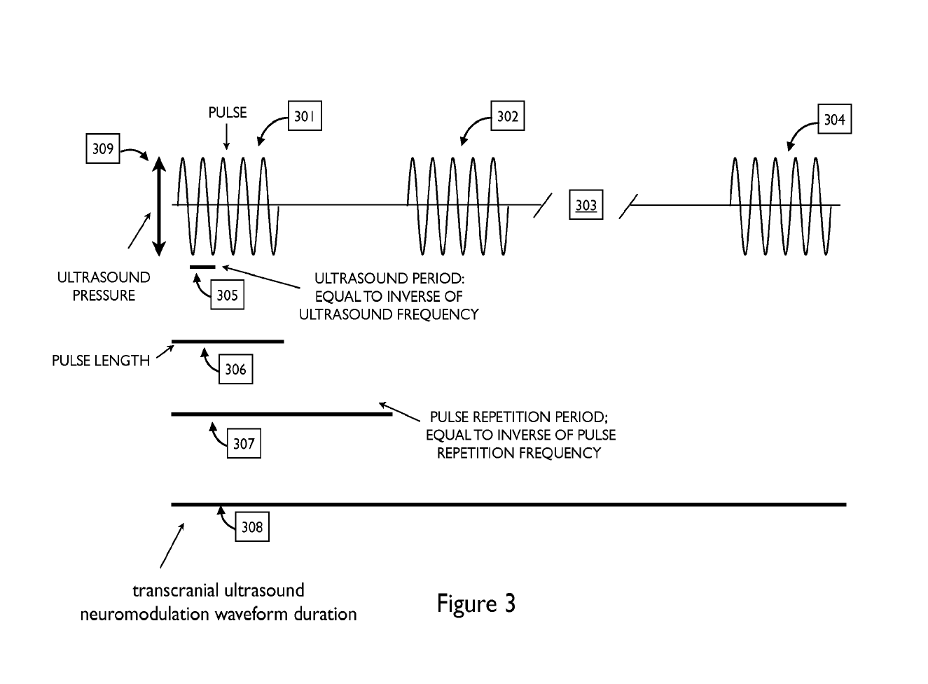

FIG. 2A shows a method 100 for transcranial ultrasound neuromodulation delivery in accordance with many embodiments. In the method 100, transcranial ultrasound neuromodulation is used to induce neuromodulation in a subject whereby:

One or more transcranial ultrasound neuromodulation ultrasound transducers are coupled to the head of an individual human or animal (the “subject”, “user”, or “recipient”) in a step 101;

1) Components of the transcranial ultrasound neuromodulation device are provided to be near or wearably attached to the recipient in order to provide power and control the intensity, timing, targeting, and waveform characteristics of the transmitted acoustic waves in a step 105;

2) a transcranial ultrasound neuromodulation protocol is triggered that uses a waveform in a step 102 that:

a. is provided with an acoustic frequency between about 100 kHz and about 10 MHz in a step 103; and

b. is provided with a spatial-peak, temporal-average intensity between about 0.0001 mW/cm² and about 10 W/cm² in a step 104; and

c. is provided with properties in a step 106 such that the waveform does not induce heating of the brain due to transcranial ultrasound neuromodulation that exceeds about 2 degrees Celsius for more than about 5 seconds; and

3) the transcranial ultrasound neuromodulation protocol induces an effect on neural circuits in one or more brain regions in a step 107.

US can cause the local vibration of particles, leading to both mechanical and thermal effects. In some embodiments, transcranial ultrasound neuromodulation brain stimulation protocols modulate neuronal activity primarily through mechanical means. In some embodiments for transcranial ultrasound neuromodulation, a single ultrasound pulse is delivered that may be referred to as a continuous wave (CW) pulse by one skilled in the art and extends in time for about longer than 10 ms, about longer than 100 ms, about longer than 1 second, or any length of time up to and including 5 seconds. Complex transcranial ultrasound neuromodulation waveforms, including transcranial ultrasound neuromodulation waveforms generated by hybridization, convolution, addition, subtraction, phase shifting, concatenation, and joining with an overlap for a portion of each of the waveforms for two or more transcranial ultrasound neuromodulation waveforms or transcranial ultrasound neuromodulation waveform components, as well as modulation or ramping of the intensity of all or a portion of the waveform, or modulation or ramping of any other parameter used to define an ultrasound waveform, may be advantageous for transcranial ultrasound neuromodulation in some embodiments.

Appropriate transcranial ultrasound neuromodulation protocols can be advantageous for mitigating or eliminating tissue damage while simultaneously modulating neuronal activity primarily through mechanical means in at least some embodiments. For example, low temporal average intensity can be achieved by reducing the acoustic power of the ultrasound waves or by varying one or more transcranial ultrasound neuromodulation parameters to decrease the effective duty cycle—the proportion of time during a transcranial ultrasound neuromodulation waveform that ultrasound is delivered. Reduced duty cycles can be achieved by decreasing one or more transcranial ultrasound neuromodulation parameters chosen from pulse length, cycles per pulse, pulse repetition frequency, or other waveform parameters. Low temporal average intensity can be achieved by varying one or more ultrasound parameters during a transcranial ultrasound neuromodulation protocol. For instance, the acoustic power may be decreased during a portion of a transcranial ultrasound neuromodulation protocol. Alternatively, the pulse repetition frequency can be decreased during a transcranial ultrasound neuromodulation protocol. In other embodiments, complex ultrasound waveforms can be generated that are effective for inducing neuromodulation and maintain an appropriately low temporal average intensity.

In some embodiments, the effect of transcranial ultrasound neuromodulation on brain function is detected by one or more technique selected from the group that includes, but is not limited to: (i) subjectively by the recipient as a perception, movement, concept, instruction, other symbolic communication by modifying the recipient's cognitive, emotional, physiological, attentional, or other cognitive state; (ii) through physiological measurement of brain activity by one or a plurality of: electroencephalography (EEG), magnetoencephalography (MEG), functional magnetic resonance imaging (fMRI), functional near-infrared spectroscopy (fNIRS), positron emission tomography (PET), single-photon emission computed tomography (SPECT), computed tomography (CT), functional tissue pulsatility imaging (fTPI), and other techniques for measuring brain activity known to one skilled in the art; and (iii) by making a physiological measurement of the body such as by electromyogram (EMG), galvanic skin response (GSR), heart rate, blood pressure, respiration rate, pupil dilation, eye movement, gaze direction, and other physiological measurement. In further embodiments, the transcranial ultrasound neuromodulation assembly further comprises one or more appropriate sensors, transducers, electrical control circuitry, signal processing systems or any combination thereof, configured to achieve one or more of the above listed techniques for measuring the physiology or brain activity of the user.

In some embodiments, a transcranial ultrasound neuromodulation protocol delivers ultrasound to one or more brain regions and induces neuromodulation that correlates more strongly in time with the timecourse of mechanical effects on tissue than thermal effects. The acoustic frequency for transcranial ultrasound neuromodulation is generally greater than about 100 kHz and less than about 10 MHz—i.e. generally greater than about 100 kHz and less than about 10 MHz; optionally greater than about 0.3 MHz and less than about 0.8 MHz; optionally greater than about 0.3 MHz and less than about 1 MHz; optionally greater than about 0.3 MHz and less than about 0.5 MHz; optionally greater than about 0.3 MHz and less than about 0.4 MHz; optionally greater than about 0.3 MHz and less than about 0.6 MHz; optionally greater than about 0.3 MHz and less than about 10 MHz; optionally greater than about 0.25 MHz and less than about 0.8 MHz; optionally greater than about 0.25 MHz and less than about 1 MHz; optionally greater than about 0.25 MHz and less than about 0.5 MHz; optionally greater than about 0.25 MHz and less than about 0.4 MHz; optionally greater than about 0.25 MHz and less than about 0.6 MHz; optionally greater than about 0.25 MHz and less than about 10 MHz; optionally greater than about 0.1 MHz and less than about 0.8 MHz; optionally greater than about 0.1 MHz and less than about 1 MHz; optionally greater than about 0.1 MHz and less than about 0.5 MHz; optionally greater than about 0.1 MHz and less than about 0.4 MHz; optionally greater than about 0.1 MHz and less than about 0.6 MHz; optionally greater than about 0.1 MHz and less than about 10 MHz; optionally greater than about 0.5 MHz and less than about 0.8 MHz; optionally greater than about 0.5 MHz and less than about 1 MHz; optionally greater than about 0.5 MHz and less than about 0.55 MHz; optionally greater than about 0.5 MHz and less than about 0.7 MHz; optionally greater than about 0.5 MHz and less than about 0.6 MHz; optionally greater than about 0.5 MHz and less than about 10 MHz; optionally greater than about 0.7 MHz and less than about 0.8 MHz; optionally greater than about 0.7 MHz and less than about 1 MHz; optionally greater than about 0.7 MHz and less than about 0.75 MHz; or optionally greater than about 0.5 MHz and less than about 10 MHz. Particularly advantageous acoustic frequencies may be between about 0.3 MHz and about 0.7 MHz.

In ultrasound, acoustic intensity can be a measure of power per unit of cross sectional area (e.g. mW/cm²) and may require averaging across space and time. The intensity of the acoustic beam can be quantified by several metrics that differ in the method for spatial and temporal averaging. These metrics can be defined according to technical standards established by the American Institute for Ultrasound in Medicine and National Electronics Manufacturers Administration (NEMA. Acoustic Output Measurement Standard For Diagnostic Ultrasound Equipment (National Electrical Manufacturers Association, 2004)). A commonly used intensity index is the “spatial-peak, temporal-average” intensity (Iₛₚₜₐ). The intensities reported herein generally refer to Iₛₚₜₐ at the targeted brain region. The spatial-peak temporal-average (Iₛₚₜₐ) intensity of the ultrasound wave in brain tissue may be greater than about 0.0001 mW/cm² and less than about 10 W/cm², i.e. generally from 21 mW/cm² to 0.1 W/cm²; optionally from 21 mW/cm² to 0.5 W/cm²; optionally from 21 mW/cm² to 1 W/cm²; optionally from 50 mW/cm² to 0.1 W/cm²; optionally from 50 mW/cm² to 0.5 W/cm²; optionally from 50 mW/cm² to 1 W/cm²; optionally from 0.1 W/cm² to 0.2 W/cm²; optionally from 0.1 W/cm² to 0.5 W/cm²; and optionally from 0.1 W/cm² to 1 W/cm². Particularly advantageous Iₛₚₜₐ values may be between about 100 mW/cm² and about 700 mW/cm², usually in the range from about 200 mW/cm² to about 500 mW/cm². The Iₛₚₜₐ value for any particular transcranial ultrasound neuromodulation protocol can be calculated according to methods well known in the art that relate to the ultrasound pressure and temporal average of the transcranial ultrasound neuromodulation waveform over its duration. Effective ultrasound intensities for activating neurons or neuronal circuits do not cause tissue heating greater than about 2 degrees Celsius, usually less than 1 degree Celsius, for a period longer than about 5 seconds, preferably no longer than 3 seconds.

Significant attenuation of ultrasound intensity can occur at the boundaries between skin, skull, dura, and brain due to impedance mismatches, absorption, and reflection so the required ultrasound intensity delivered to the skin or skull may exceed the intensity at the targeted brain region by up to 10-fold or more depending on skull thickness and other tissue and anatomical properties.

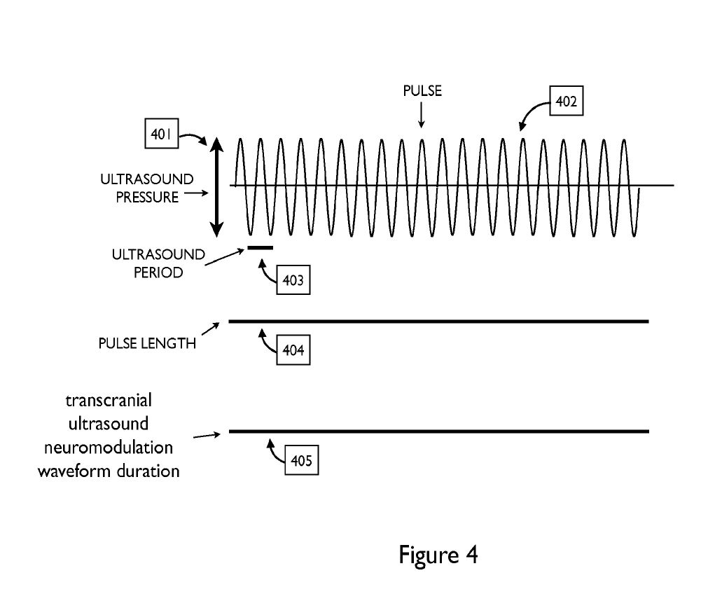

FIG. 3 shows a transcranial ultrasound neuromodulation waveform and a pulsed ultrasound protocol, in accordance with many embodiments. Pulsing of ultrasound can be an effective means for activating neurons that reduces the temporal average intensity while also achieving desired brain stimulation or neuromodulation effects. In addition to acoustic frequency and transducer variables, several waveform characteristics such as cycles per pulse, pulse repetition frequency, number of pulses, and pulse length affect the intensity characteristics and outcome of any particular transcranial ultrasound neuromodulation stimulus on brain activity. A pulsed transcranial ultrasound neuromodulation protocol generally uses pulse lengths between about 0.5 microseconds and about 1 second, i.e. generally from 0.5 microseconds to 5 microseconds; optionally from 0.5 microseconds to 50 microseconds; optionally from 0.5 microseconds to 100 microseconds; optionally from 0.5 microseconds to 500 microseconds; optionally from 0.5 microseconds to 1 ms; optionally from 0.5 microseconds to 10 ms; optionally from 0.5 microseconds to 100 ms; optionally from 0.5 microseconds to 500 ms; optionally from 0.5 microseconds to 1 second; optionally from 5 microseconds to 50 microseconds; optionally from 5 microseconds to 100 microseconds; optionally from 5 microseconds to 500 microseconds; optionally from 5 microseconds to 1 ms; optionally from 5 microseconds to 10 ms; optionally from 5 microseconds to 100 ms; optionally from 5 microseconds to 500 ms; optionally from 5 microseconds to 1 second; optionally from 100 microseconds to 500 microseconds; optionally from 100 microseconds to 1 ms; optionally from 100 microseconds to 10 ms; optionally from 100 microseconds to 100 ms; optionally from 100 microseconds to 500 ms; optionally from 100 microseconds to 1 second; optionally from 500 microseconds to 1 ms; optionally from 500 microseconds to 10 ms; optionally from 500 microseconds to 100 ms; optionally from 500 microseconds to 500 ms; optionally from 500 microseconds to 1 second; optionally from 1 ms to 10 ms; optionally from 1 ms to 100 ms; optionally from 1 ms to 500 ms; optionally from 1 ms to 1 second; and optionally from and 100 ms to 1 second. A transcranial ultrasound neuromodulation protocol may use pulse repetition frequencies (PRFs) between about 50 Hz and about 25 kHz, i.e. generally from 50 Hz to 100 Hz; optionally from 50 Hz to 250 Hz; optionally from 50 Hz to 1 kHz; optionally from 50 Hz to 2 kHz; optionally from 50 Hz to 3 kHz; optionally from 50 Hz to 4 kHz; optionally from 50 Hz to 5 kHz; optionally from 50 Hz to 10 kHz; optionally from 50 Hz to 25 kHz; optionally from 100 Hz to 250 Hz; optionally from 100 Hz to 1 kHz; optionally from 100 Hz to 2 kHz; optionally from 100 Hz to 3 kHz; optionally from 100 Hz to 4 kHz; optionally from 100 Hz to 5 kHz; optionally from 100 Hz to 10 kHz; optionally from 100 Hz to 25 kHz; optionally from 250 Hz to 500 Hz; optionally from 250 Hz to 1 kHz; optionally from 250 Hz to 2 kHz; optionally from 250 Hz to 3 kHz; optionally from 250 Hz to 4 kHz; optionally from 250 Hz to 5 kHz; optionally from 250 Hz to 10 kHz; optionally from 250 Hz to 25 kHz; optionally from 500 Hz to 1 kHz; optionally from 500 Hz to 2 kHz; optionally from 500 Hz to 3 kHz; optionally from 500 Hz to 4 kHz; optionally from 500 Hz to 5 kHz; optionally from 500 Hz to 10 kHz; optionally from 500 Hz to 25 kHz; optionally from 1 kHz to 2 kHz; optionally from 1 kHz to 3 kHz; optionally from 1 kHz to 4 kHz; optionally from 1 kHz to 5 kHz; optionally from 1 kHz to 10 kHz; optionally from 1 kHz to 25 kHz; optionally from 3 kHz to 4 kHz; optionally from 3 kHz to 5 kHz; optionally from 3 kHz to 10 kHz; optionally from 3 kHz to 25 kHz; optionally from 5 kHz to 10 kHz; optionally from 5 kHz to 25 kHz; and optionally from and 10 kHz to 25 kHz. Particularly advantageous PRFs may be generally between about 1 kHz and about 3 kHz. For pulsed transcranial ultrasound neuromodulation waveforms, the number of cycles per pulse (cpp) may be between about 5 and about 10,000,000. Particularly advantageous cpp values can vary depending on the choice of other transcranial ultrasound neuromodulation parameters and are generally between about 10 and about 250. The number of pulses for pulsed transcranial ultrasound neuromodulation waveforms may be between about 1 pulse and about 125,000 pulses. In FIG. 3, the 1st (301), 2nd (302), and nth (304) pulses are shown, with the gap in the horizontal line (303) indicating additional pulses that may number between about 1 and about 125,000 pulses. The waveform has a pulse wavelength 305, a pulse length 306, a pulse repetition period 307, and a total waveform duration 308. Particularly advantageous pulse numbers for pulsed transcranial ultrasound neuromodulation waveforms may be between about 100 pulses and about 250 pulses.

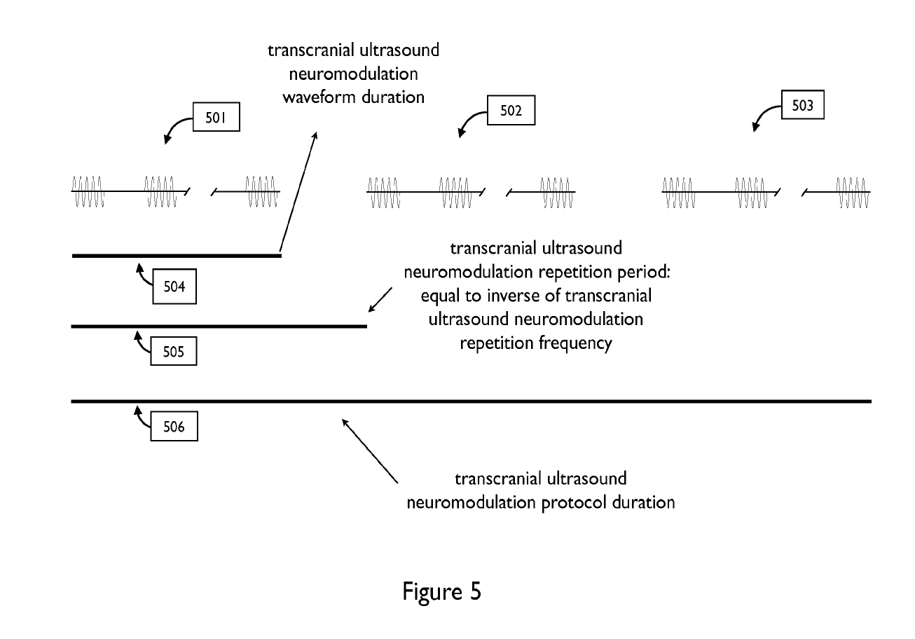

FIG. 4 shows a transcranial ultrasound neuromodulation waveform, and a continuous wave ultrasound protocol, in accordance with many embodiments. Tone bursts of ultrasound energy that extend for about 1 second or longer (such as pulse 402 having an amplitude 401, a wavelength 403, a pulse length 404, and a waveform duration 405 shown in FIG. 4)—though, strictly speaking, also pulses—are often referred to as continuous wave (CW). In alternative embodiments, one or more continuous wave (CW) ultrasound waveforms are less than about five seconds in duration, typically being from 1 second to 5 seconds. US protocols that include such CW waveforms offer advantages for neuromodulation due to their capacity to drive activity robustly. In at least some cases, transcranial ultrasound neuromodulation protocols with CW pulses may have temporal average intensities that can be significantly higher which may cause painful thermal stimuli on the scalp or skull and may also induce heating and thus damage in brain tissue. Thus, embodiments disclosed herein using CW pulses may employ a lower acoustic intensity and/or a slow pulse repetition frequency of less than about 1 Hz. For instance, a CW US stimulus waveform with 1 second pulse lengths repeated at 0.5 Hz would deliver US every other second. Alternative pulsing protocols including those with slower pulse repetition frequencies of less than about 0.5 Hz or less than about 0.1 Hz or less than about 0.01 Hz or less than about 0.001 Hz may also be beneficial. In some embodiments, the interval between pulses or pulse length may be varied during a transcranial ultrasound neuromodulation protocol that includes CW pulses.

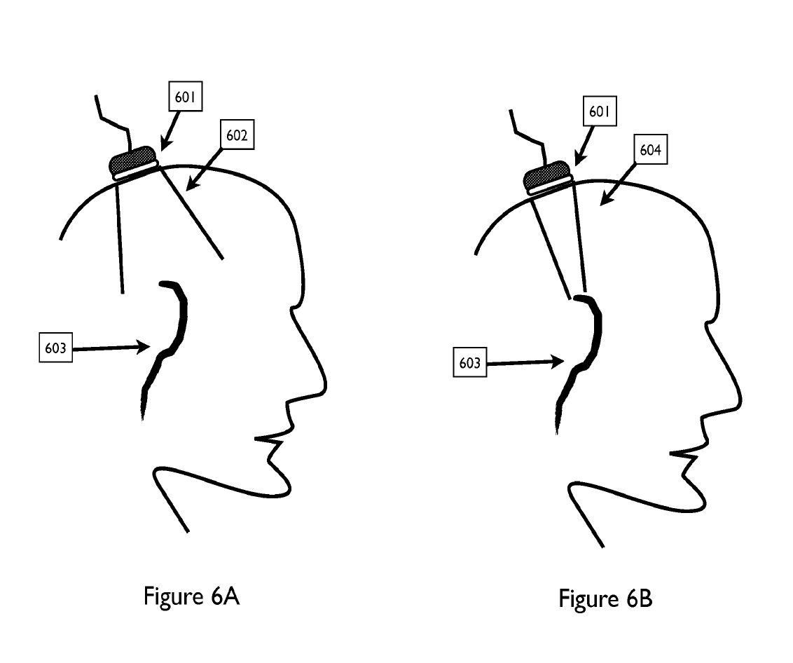

FIG. 5 shows transcranial ultrasound neuromodulation waveform repetition, in accordance with many embodiments. In some embodiments, repeating a transcranial ultrasound neuromodulation protocol is advantageous for achieving particular forms of neuromodulation during a transcranial ultrasound neuromodulation session. In some embodiments, the number of times a transcranial ultrasound neuromodulation protocol of appropriate duration 504 is repeated is chosen to be in the range between 2 times and 100,000 times. FIG. 5 presents a schematic of three repeated transcranial ultrasound neuromodulation protocols (501, 502, 503) that together represent a transcranial ultrasound neuromodulation protocol 506. Particularly advantageous numbers of transcranial ultrasound neuromodulation protocol repeats may be between 2 and 1,000 repeats. Repetition frequency 505 of a transcranial ultrasound neuromodulation protocol may be less than about 10 Hz, less than about 1 Hz, less than about 0.1 Hz, or lower. The transcranial ultrasound neuromodulation repetition frequency may be fixed or variable. Variable transcranial ultrasound neuromodulation repetition frequency values may be random, pseudo-random, ramped, or otherwise modulated. The transcranial ultrasound neuromodulation repetition period can be defined as the inverse of the transcranial ultrasound neuromodulation repetition frequency.

More complex transcranial ultrasound neuromodulation waveforms can also be generated using one or more programmable function generators. Alternatively, complex waveforms are generated with appropriate software such as Matlab (Mathworks, Natick, Mass.) or LabVIEW (National Instruments, Austin, Tex.), then communicated by electronic components via a wired or wireless communication protocol to one or more components of the system that transduce ultrasound acoustic waves and couple them to the subject transcranially.

Various ultrasound transducers can be used to generate the acoustic wave for transcranial ultrasound neuromodulation. Specific water immersion type transducers include the Ultran GS500-D13, NDT Systems IBMF0.53, Ultran GS350-D19, Olympus Panametrics V318 focused transducer 0.5 MHz/0.75″ F=0.85″, Ultran GS200-D25 and Olympus Panametrics V301S 0.5 MHz/1.0″. Customized ultrasound transducers designed with appropriate intensity and resonant acoustic frequency characteristics may also be advantageous for delivering transcranial ultrasound neuromodulation. For instance, a Blatek AT21926 Rev 0 transducer tuned to 300 kHz may be beneficial for transcranial ultrasound neuromodulation.

Providing a mixture of ultrasound frequencies may be useful for efficient brain stimulation via transcranial ultrasound neuromodulation. Various strategies for achieving a mixture of ultrasound frequencies to the brain of the user may be applied. Driving an ultrasound transducer at a frequency other than the resonant frequency of the transducer may be a way to create ultrasound waves that contain power in a range of frequencies. For instance, an ultrasound transducer with a center frequency of 0.5 MHz can be driven with a sine wave at 0.35 MHz. Another means for producing ultrasound waves that contain power in a range of frequencies may be to use square or other nonsinusoidal waves to drive the transducer. Yet another means for generating a mixture of ultrasound frequencies may be to choose transducers that have different center frequencies and drive each at their resonant frequency. A further means for generating a mixture of ultrasound frequencies may be to drive an ultrasound transducer with a waveform that itself contains multiple frequency components. One or more of the above strategies or alternative strategies known to those skilled in the art for generating US waves with a mixture of frequencies may also be beneficial.

An advantageous feature of ultrasound's ability to modulate brain function may lie in its multi-frequency nature. Use of broadband ultrasound signals can be particularly efficacious for generating neuromodulatory effects. Broadband ultrasound that contains multiple frequency components, including frequencies low compared to the incident ultrasound frequency, can be created locally within tissue by the combined use of ultrasound and acoustic contrast agents. This form of broadband ultrasound can even be generated by single-frequency ultrasound when combined with acoustic contrast agents.

In an alternative embodiment of the invention, acoustic contrast agents are used to deliver broad-band ultrasound of appropriate frequencies for transcranial ultrasound neuromodulation. Acoustic contrast agents typically comprise stable bubbles filled with compressible fluids or, more typically, gas, coated with a variety of materials to help facilitate their stabilization after they are injected into the blood stream. The bubbles can change their volume due to overpressure caused by the ultrasound impinging upon the bubbles, at least at the frequency of the applied ultrasound. The bubbles may push upon the surrounding fluid due to their change in volume, thereby generating pressure waves within the surrounding blood that propagate away from the bubble. The pressure waves in the blood—and the tissue that surrounds the blood vessels—will typically be as spectrally complex as the bubble motion itself. For low applied pressures, the bubbles can tend to produce acoustic emissions at the same frequency as ultrasound that stimulates the bubbles. At higher pressures the bubbles may experience more complex volumetric changes as well as changes in shape. The result can be local production of broadband ultrasound energy emitted by the bubbles and absorbed into surrounding tissue.

When the ultrasound and bubble characteristics are tuned appropriately, they can produce a desired local ultrasound field with appreciable spectral complexity, thereby creating a spatially restricted neuromodulatory effect where the ultrasound (even single-frequency ultrasound) overlaps with the presence of bubbles of acoustic contrast agents.

In some embodiments, transcranial ultrasound neuromodulation can be delivered from a phased array of transducers for improved targeting of one or more brain regions. Constructive and destructive interference of acoustic waves transmitted by multiple transducers can be used to deliver complex spatiotemporal patterns of acoustic waves. Moreover, the spectral density of acoustic pressure profiles delivered to a targeted brain region can be varied to produce differential effects on neuronal activity. These properties of transcranial ultrasound neuromodulation offer the possibility of activating widely distributed brain networks. In certain embodiments, the capacity to target distributed brain regions concurrently or with a specific order further can extend the possibilities for modulating brain activity. In an alternative embodiment, a plurality of ultrasound transducers are employed for delivering transcranial ultrasound neuromodulation to a subject and the transcranial ultrasound neuromodulation waveform delivered from some or all ultrasound transducers differs in one or a plurality of parameters that may include intensity, acoustic frequency, pulse duration, pulse repetition frequency, ultrasound cycle shape, or another parameter that defines the transcranial ultrasound neuromodulation waveform. Similarly, in some embodiments, aTCD can be delivered from a phased array of transducers for improved imaging of brain blood vessels in one or more brain regions.

In alternative embodiments, multiple ultrasound transducers operating at higher acoustic frequencies (in a range from about 1 MHz to about 15 MHz; particularly advantageous frequencies being between about 1 MHz and about 3 MHz) can be used to achieve transcranial ultrasound neuromodulation via vibroacoustography stimulation. In these embodiments, systems operate by running two or more transducer elements in a coordinated fashion with two or more ultrasound frequencies such that they can produce frequency-modulated signals at one or more foci at frequencies advantageous for transcranial ultrasound neuromodulation of about 0.1 MHz to about 1 MHz. At the site of cells to be modulated, the interference of acoustic frequencies generates vibratory mechanical effects with a dominant frequency equal to the difference of the ultrasound waveforms delivered transcranially.

For the vast majority of transducers (air-coupled transducers being an exception), the ultrasound device must be in physical contact with the subject due to the poor impedance match between air and tissue. Ultrasound gel (or another coupling material) is usually used to couple the transducer apparatus to the head to minimize distortion or reflection of the ultrasound waveform due to acoustic impedance mismatch. In some embodiments of a transcranial ultrasound neuromodulation and aTCD device, components for cooling are used due to heating that can occur in the transducer, coupling gel, brain, and/or body. Although some components of the transcranial ultrasound neuromodulation and aTCD device may be placed remotely from the subject, transducers other than air-coupled transducers require physical attachment to the subject in this embodiment. The subject's head may be placed in an assembly that holds the transducer assembly in contact with the user. Alternatively, the transducer apparatus may be wearably attached to the user with a helmet, headband, adhesive material, hat, eyeglasses, or other piece of wearable hardware or clothing. In alternative embodiments, a transducer apparatus is held in place on the head, for instance by using a hand.

Several strategies are known for focusing ultrasound waves to a specific brain region. These strategies are advantageous for restricting the area of both neuromodulation via transcranial ultrasound neuromodulation and blood vessel imaging via aTCD. The lateral extent of the spatial envelope of US transmitted into the brain can be restricted by using acoustic collimators. Single-element transducers having concave focusing lenses or transducers shaped to deliver a targeted acoustic wave can also be used for delivering focused acoustic pressure fields to brains. Such single-element focused transducers can be manufactured having various focal lengths depending on the lens curvature, as well as the physical size and center frequency of the transducer. The most accurate yet complicated US focusing method involves the use of multiple transducers operating in a phased array. An alternative means for focusing uses components that shift, rotate, or otherwise move one or more ultrasound transducers.

Transcranial Doppler Ultrasound

High frequency ultrasound can be employed for transcranial Doppler imaging of flow in brain blood vessels, including automated transcranial Dopper (aTCD). In some embodiments, brain blood vessel maps generated by TCD are used to establish a coordinate system for steering transcranial ultrasound neuromodulation for neuromodulatory effect. Effective acoustic frequencies for transcranial Doppler imaging may generally be in a range between about 1 MHz and about 4 MHz. Particularly advantageous frequency ranges for TCD may be from about 1.75 to about 2 MHz.

One or more components for aTCD imaging may provide: (1) affordable three-dimensional imaging of blood flow using a low-profile easily-attached transducer pad, (2) real-time vector velocity of blood flow, and (3) long-term unattended Doppler-ultrasound monitoring in spite of motion of the patient or aTCD system. The one or more components that may be useful for aTCD may achieve one or more of: (1) locating blood vessels in the brain and (2) defining a 3-dimensional map of these blood vessels.

Methods and systems for defining a 3-dimensional map of blood vessels in the brain of a human subject via aTCD have been described previously in U.S. patent application Ser. No. 12/756,108, published as U.S. Publication No. 2011/0251489 and entitled “Ultrasound Monitoring Systems, Methods and Components,” by Zhang et al.; U.S. patent application Ser. No. 11/234,914, published as U.S. Publication No. 2006/0100530 and entitled “Systems and methods for non-invasive detection and monitoring of cardiac and blood parameters,” by Kliot et al.; U.S. Pat. No. 7,547,283, entitled “Methods for Determining Intracranial Pressure Non-invasively,” by Mourad et al.; and U.S. Pat. No. 6,682,483, entitled “Device and Method for Mapping and Tracking Blood Flow and Determining Parameters of Blood Flow,” by Abend et al. Methods and systems for locating and tracking blood vessels in the brain despite movement of the user and/or one or more components of the aTCD device have also been reported previously and may be further beneficial features of a device for targeting transcranial ultrasound neuromodulation via aTCD. Published studies have disclosed an ambulatory aTCD system that requires a trained neurosonographer to find the middle cerebral artery (MCA). These systems stay locked on to the MCA if there are small motions of the TCD system but are not truly automated.

Various ultrasound transducers can be used to generate the acoustic wave for aTCD. For aTCD, advantageous single-element transducers include those deployed by Spencer Technologies and Multigon that may be attached to a mechanical device that moves the transducer to automate the TCD process, as well as two-dimensional ultrasound arrays built by PhysioSonics Inc. for the express purpose of automating TCD. Another embodiment for aTCD may use a linear array which may be similar in concept (with different underlying control systems) to so-called cardiac probes used for imaging the heart.

In many embodiments, targeting and/or focusing of transcranial ultrasound neuromodulation is guided by the relative position of (1) the one or more targeted brain regions and (2) a 3-dimensional map defined by an aTCD system of one or more brain blood vessels that serve as a landmark or fiduciary coordinate system. The following section includes descriptions of the systems and methods for locating brain blood vessels, defining a 3-dimensional map, determining the location of the one or more targeted brain regions relative to the blood vessel map, and, in some embodiments, updating the 3-dimensional map continuously or intermittently to account for movement of the user or one or more components wearably attached to the user.

One or more components used for automated transcranial Doppler (aTCD) may perform blood velocity monitoring by collecting Doppler data in three dimensions; azimuth, elevation, and range (depth); so that the point (in three dimensional space) at which the velocity is to be monitored can be acquired and tracked when the user or the sensor moves. A three dimensional map of the blood flow may also be produced and measured radial velocity may be converted to true vector velocity.

Since the targeting device of the many embodiments can automatically locate and lock onto one or a plurality of points with absolute or local maximum volume of blood having a significant radial velocity, unattended continuous blood velocity monitoring may be one of its uses. By using the precise relative location of the point at which lock occurs as a function of depth, the device can map the network of blood vessels as a 3-dimensional track without the hardware and computational complexity required to form a conventional ultrasound image. Using radial velocity along with the three-dimensional blood path, the device can directly compute vector velocity.

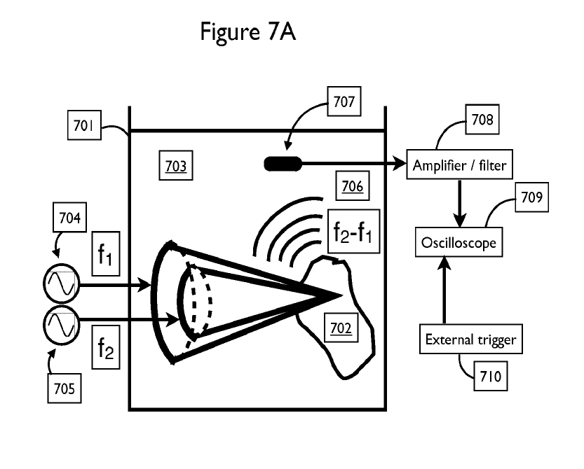

FIG. 6A illustrates, schematically, the use of a scanning acoustic transducer assembly 602 that acoustically illuminates and acquires acoustic data from multiple points within a broad target area 601, such as a large portion of the cerebral blood vessel complex, in a scanning mode. Based on the acoustic data acquired in the scanning mode, localized target sites 603 within the scanned area may be identified and elements of the transducer assembly are focused on localized target site(s) for acquisition of acoustic data from the desired target site(s), as shown in FIG. 6B. Selection of localized target site(s) may be predetermined based on various acoustic properties, including the amplitude (or any amplitude derivative) of acoustic scatter data, Doppler analysis of acoustic scatter data, phase or frequency of acoustic data, changes in the primary and/or other maxima and/or minima amplitude, phase or frequency of acoustic signals within a cardiac and/or respiratory cycle or other period, or determinations derived from acoustic data, such as flow velocity, tissue stiffness properties, endogenous and/or induced tissue displacement properties, acoustic emissions associated with such displacements, rates of change of such properties, and the like.

Various noninvasive, non-acoustic detection modalities may be employed additionally to locate internal physiological structures, including blood vessels such as the MCA, prior to acquisition of acoustic data. Near infra-red spectroscopy (NIRS), magnetic resonance imaging, and other techniques may be used, for example, to image and locate internal physiological structures. Such techniques may be used in association with the methods and systems of the present invention for locating internal physiological structures prior to assessment of acoustic properties.

In some embodiments, coordinates for target vessel volume location and values for acoustic properties may be recorded and stored, over time, and displayed in a variety of formats. A pattern defined by aTCD focused on any set of two or more blood vessels can be used to form a 3-dimensional map that permits improved initial targeting as well as maintained targeting by transcranial ultrasound neuromodulation to one or more brain regions.

In some embodiments, targeting of transcranial ultrasound neuromodulation can be modified during or between transcranial ultrasound neuromodulation sessions based on aTCD measurements. aTCD measurements may be taken continuously or intermittently to determine whether there have been relative movements of the transcranial ultrasound neuromodulation ultrasound transducers and the targeted brain region and, generally, the subject's head. In some embodiments in which intermittent aTCD measurements are used, aTCD measurements can be made about more than once per day, about more than once per 12 hours, about more than once per 6 hours, about more than once per 3 hours, about more than once per 2 hours, about more than once per 1 hour, about more than once per 30 minutes, about more than once per 15 minutes, about more than once per 10 minutes, about more than once per 5 minutes, about more than once per 3 minutes, about more than once per minute, about more than once per 30 seconds, about more than once per 15 seconds, about more than once per 10 seconds, about more than once per 5 seconds, or about more than once per second.

Using methodologies and assemblies described below, an acoustic source/detector combination, preferably an acoustic transducer array comprising multiple transducer elements, can be operable in both a scanning mode and a focusing mode. One or more acoustic source element(s) of the acoustic data acquisition component may acoustically illuminate a relatively broad desired target area in a scanning mode to identify target sites having predetermined or desired acoustic properties, thus identifying the target site(s) as blood vessel(s). When the acoustic source has identified one or more target sites having the predetermined or desired acoustic properties, one or more of the acoustic source(s) may be manually or automatically focused on the desired target site(s) for operation in an acoustic interrogation or data acquisition mode. The acoustic source may also be programmed to monitor acquired acoustic data and to adjust the positioning and/or focus of the source to maintain the focus of selected or predetermined acoustic source(s) on the desired target site. Similarly, acoustic source(s) may be programmed to acquire data from a plurality of predetermined or programmed target sites at predetermined time points.

Having identified the location of the one or more target vessels in a scanning mode, one or more target vessel volumes may be selected for data acquisition and analysis. For methods and systems involving data acquisition from the middle cerebral artery (MCA), as described above, the acoustic focus and data acquisition volume generally represents substantially the entire cross-section of the target MCA vessel.

Acoustic frequencies of from about 0.5 MHz to 15 MHz, more preferably from about 1 MHz to about 10 MHz, most preferentially between about 1 MHz and about 4 MHz, or generally about 1 MHz to about 2 MHz may be used for monitoring blood flow in vessels in the brain, with intensities as measured in water conforming to FDA guidelines, typically less than about 1 W/cm², pulse repetition frequency generally between about 0.5 kHz to about 10 kHz, typically about 1 kHz to about 8 kHz, pulse duration generally about 1 microsecond to about 200 microseconds, most typically about 5 microseconds to about 40 microseconds. Preferable acoustic frequencies for aTCD may be in a range from about 1 MHz to about 3 MHz. Higher acoustic frequencies may be poorly transmitted through the skull and thus less preferable for aTCD. In other embodiments, ultrasound is transmitted through the occipital foramen to provide high resolution acoustic data with a generally low level of artifacts. Vessel monitoring may also be accomplished using multiple frequencies for acoustically interrogating and/or for acoustic data acquisition over time and/or over vessel sample volumes to facilitate enhanced detection of blood flow parameters and anomalies. Acoustic transducer source and detector elements of the present invention may, in fact, be programmed to collect one or more types of acoustic data from a single or multiple target sites, at one or more frequencies and at one or more times. Acquisition of acoustic data, using methods and systems of the present invention, may be preferably accomplished in an automated fashion.

In some embodiments where angular resolution based on wavelength and aperture size is inadequate, fine mapping may be achieved, for example, by post-Doppler monopulse tracking each range cell of each vessel, and recording the coordinates describing the location of the monopulse null. With a three-dimensional map available, true vector velocity can be computed. For accurate vector flow measurement, the monopulse difference can be computed in a direction orthogonal to the vessel by digitally rotating until a line in the azimuth-elevation or C-scan display is parallel to the vessel being monitored.

In some embodiments, post-Doppler, sub-resolution tracking and mapping is utilized: Doppler processing may be done first using only high Doppler-frequency data. This results in extended targets since the active vessels approximate “lines” as opposed to “points”. In three-dimensional space, these vessels can be resolved, one from another. At a particular range, the azimuth-elevation axis can be rotated so that the “line” becomes a “point” in the azimuth dimension. That point can then be located by using super-resolution techniques or by using a simple technique such as monopulse.

In different embodiments, various types of acoustic transducers and acoustic transducer arrays may be used as acoustic source/detector assemblies and acoustic data acquisition components. A single acoustic transducer, or a single acoustic transducer array may be operated both as a source and a detector, or separate source and detector transducers or transducer arrays may be provided. Conventional piezoelectric (PZT) acoustic transducers may be implemented as acoustic data acquisition components in methods and systems of the present invention. Acoustic transducer arrays composed of capacitive micromachined ultrasonic transducers (cMUTs) and polyvinylidene fluoride (PVDF) cells or elements may also be used and are preferred for many implementations. PZT, cMUT, and PVDF acoustic transducers and arrays may be combined in various data acquisition components and operated in acoustic source and/or receiver modes in yet other embodiments. Two advantageous features of PVDF and cMUT transducers are that they are very broad band and disposable. In some embodiments, PVDF transducers are more effective as receivers only for aTCD due to their low source power.

In some embodiments of the TCD component of the apparatus, methods and systems are used for locating and acoustically illuminating and/or probing a desired brain blood vessel target site in an automated fashion. An acoustic transducer/receiver array may be employed in a scanning mode, for example, to acquire acoustic data from numerous sites within a larger target area. Based on the acoustic data collected in the scanning mode, localized sites within the target area may be selected as target sites for focused acoustic illumination and/or probing. Localized target sites may be selected, or predetermined, based on any aspect of the acoustic data collected in the scanning mode, such as acoustic scatter amplitude, phase and/or frequency maxima or minima, tissue stiffness properties, endogenous and/or induced tissue displacement properties, rates of change of such properties, and the like. In another embodiment, an automated system is provided that locates a desired target site within a larger target area in a scanning mode, focuses on the desired target site for acquisition of acoustic data, and thereafter periodically scans the target area and repositions the acoustic focus, if necessary, to maintain the focus of the acoustic source at the desired target site. Multiple target sites may also be located in a scanning mode and focused on sequentially and/or simultaneously for acoustic data acquisition from multiple target sites using acoustic transducer/receiver array assemblies.

Methodologies for scanning and locating desired brain blood vessel target areas based on their acoustic properties may be based on “range-Doppler” search methodologies. Range-Doppler processing may be an efficient implementation of matched filtering that has been used in the radar and sonar signal processing community for many years. It can be a robust technique, in part because it makes very few assumptions about the statistical nature of the environment and targets that it encounters. “Range-Doppler” and other methodologies for finding and maintaining an acoustic focus on a desired target area may also be applicable, including those described in U.S. Pat. No. 7,547,283, entitled “Methods for Determining Intracranial Pressure Non-invasively,” by Mourad et al., and other techniques known in the art.

In other embodiments, the user or another individual has the option to assist the automated targeting. This may be useful, for example, for cases where systems for automatically identifying the brain blood vessel feature of interest may not be uniquely converging on that feature, or so that the user or other individual can validate whether or not the brain blood vessel feature chosen by the computer is, in their opinion, the optimal feature. In some embodiments, automated targeting can be further improved by integrating TCD imaging with other techniques for imaging in the brain, including MRI, CT scan, and other techniques for imaging vascular and other tissue in the brain.

TCD devices also allow for the device to emit sound or use a visual display to communicate an aspect of the aTCD data, such as flow velocity or targeting of the aTCD device. In some embodiments, the user may be guided for manual placement of the aTCD device by an arrow or other visual indicator shown on a display.

Various data processing techniques may be used to condition acquired acoustic data. These include, for example, downsampling and/or resampling of telemetry and Doppler flow data to provide that each linear signal record occupies the same amount of space so that standard signal processing techniques may be employed more easily. Data cleaning may also be implemented to ensure that all signal records are continuous, within expected physiologic ranges, and appropriate for further processing. Anomalies may trigger an alarm or notification to provide monitoring information and alert the user or a monitoring professional that the data acquisition device is no longer operating properly. Phase alignment of cardiac cycle boundaries may be generally implemented to ensure the input data is in phase with regard to cardiac cycle boundaries.

If pulse-domain transformation is performed, the data may require alignment, such as through cross-correlation spectrum analysis or other methodologies as described, for example, in U.S. patent application Ser. No. 11/234,914, published as U.S. Publication No. 2006/0100530 and entitled “Systems and Methods for Non-invasive Detection and Monitoring of Cardiac and Blood Parameters,” by Kliot et al.

The pattern of ultrasound waves transmitted and post-processing of Doppler signals can take various forms in different embodiments of the invention. Various embodiments of ultrasound waveforms and signal processing for aTCD are described in U.S. Pat. No. 6,682,483, entitled “Device and Method for Mapping and Tracking Blood Flow and Determining Parameters of Blood Flow,” Abend et al. The features described therein that may be applicable to the aTCD function of the present systems, devices, and methods in some embodiments include but are not limited to:

In some embodiments, the one or more components used for aTCD also utilize (1) array thinning with large elements and limited scanning, (2) array shapes to reduce peak sidelobes and extend the field of coverage, (3) post-Doppler sub-resolution tracking, (4) post-Doppler sub-resolution mapping, (5) additional methods for maximizing the angular field of view, and (6) various digital beamforming procedures for implementing the mapping, tracking, and measurement processes. The present systems, devices, and methods also extend to array thinning, where the separation between array elements is significantly larger than half the wavelength. This can reduce the number of input cables and input signals to be processed while maintaining high resolution and sensitivity and avoiding ambiguities. In a transcranial Doppler application where signal to noise and hence receiver array, area may be highly importance, and array thinning may be possible without reducing the receiver array area because a relatively small (compared to other applications) angular field of view may be needed.

Once a section of a blood vessel is resolved from other vessels in Doppler, depth, and two angles (azimuth and elevation), post-Doppler sub-resolution processing can locate that section to an accuracy that is one-tenth to one-twentieth of the resolution. This can allow for precise tracking and accurate mapping. Tracking provides for the possibility of unattended long term monitoring and mapping aids the operator in selecting the point or points to be monitored.

Methods for extending the angular field of view of the thinned array (that is limited by grating lobes) may include (1) using multiple panels of transducers with multiplexed processing channels, (2) convex V-shaped transducer panels, (3) cylindrical shaped transducer panel, (4) spherical shaped transducer panel, and (5) negative ultrasound lens. If needed, moving the probe and correlating the sub-images can create a map of an even larger region. Reduction of grating lobes due to array thinning can be achieved by using wide bandwidth and time delay steering.

In the tracking mode, a few receiver beams may be formed at a time: sum, azimuth difference, elevation difference, and perhaps, additional difference beams, at angles other than azimuth (=0 degrees) and elevation (=90 degrees). Monopulse can be applied at angles other than 0 and 90 degrees (for example 0, 45, 90, and 135 degrees) in order to locate a vessel in a direction perpendicular to the vessel.

Improved resolution can be achieved by “super-resolution” or “parametric” techniques used in “modern spectral estimation”, including the MUSIC algorithm and autoregressive modeling, for example, SRA flow or other Doppler processing or post-processing techniques known in the art may allow an extremely accurate map of 3-D flow.

Specialized framework components may be provided for mounting to and stable positioning on different portions of a subject's anatomy and are designed with one or more integral or detachable probe mount(s) for receiving an ultrasound transducer housing, or probe, and positioning the probe in proximity to an anatomical surface of a subject, such as a skin surface. Bands or similar components may be provided to at least partially underlie the framework component, providing a comfortable interface with a subject's anatomical surface and providing an effective mounting surface for a framework component. In some embodiments, the acoustic source/detector combination may be mounted on a stabilizer, or on or in a structure, such as a helmet-type structure or headband that may be mounted on the head, for example, as shown with annular array 12a in FIG. 1B. An applicator containing an acoustically transmissive material, such as an acoustic gel, may be placed between the surface of the acoustic source/detector combination and the head. Steering of the acoustic device may be accomplished manually or using automated mechanisms, such as mechanical or electronic steering mechanisms.

Beneficial embodiments may target transcranial ultrasound neuromodulation to one or more brain regions by using information provided by aTCD components of the system. In various embodiments, the one or more brain regions targeted mediate sensory experience, motor performance, and the formation of ideas and thoughts, as well as states of emotion, physiological arousal, sexual arousal, attention, creativity, relaxation, empathy, connectedness, and other cognitive states. In some embodiments, delivering transcranial ultrasound neuromodulation to modulate neuronal activity underlying multiple sensory domains and/or cognitive states concurrently or in close temporal arrangements would be beneficial.

The capacity for targeting any brain region non-invasively may be a beneficial aspect of transcranial ultrasound neuromodulation. Due to the effective transmission of ultrasound waves through tissue, transcranial ultrasound neuromodulation permits neuromodulation throughout the brain. Distinct brain regions may be known to mediate specific cognitive functions. Other aspects of brain function can be highly distributed. One or more brain regions may be targeted concurrently to achieve the desired neuromodulatory effect for the user.

In some embodiments of transcranial ultrasound neuromodulation, ultrasound waves are targeted to areas of the cerebral cortex. The cerebral cortex is composed of four lobes: the frontal, parietal, occipital, and temporal lobes. The frontal lobe is believed to underlie motor planning, motor control, executive control, decision-making, pain-processing, social cognition, and many other higher cognitive functions. Sub-regions of frontal cortex have been identified that underlie these and other specific processes. The parietal lobe is believed to be involved in sensory processing, some aspects of motor control such as gaze control, and a variety of other functions. The occipital lobe is believed to be primarily involved in visually processing. The temporal lobe may mediate auditory processing, many aspects of language production and reception, and important aspects of long-term memory. Various regions of cerebral cortex may be sensory processing areas, including: striate visual cortex, visual association cortex, primary and secondary auditory cortex, somatosensory cortex, primary motor cortex, supplementary motor cortex, premotor cortex, the frontal eye fields, prefrontal cortex, orbitofrontal cortex, dorsolateral prefrontal cortex, ventrolateral prefrontal cortex, and anterior cingulate cortex. Transcranial ultrasound neuromodulation targeted to one or more of the above listed regions of cerebral cortex can modulate related cognitive processes or motor commands by activating, inhibiting, or otherwise modulating the function of neuronal circuits.

In other embodiments of transcranial ultrasound neuromodulation, deeper brain regions are targeted. A non-exhaustive list of brain regions that may be targeted includes: the limbic system (including the amygdala), hippocampus, parahippocampal formation, entorhinal cortex, subiculum, thalamus, hypothalamus, white matter tracts, brainstem nuclei, cerebellum, or other brain region. An alternative embodiment employs a means of targeting brain regions underlying the function of a neuromodulatory system.

Another embodiment of transcranial ultrasound neuromodulation to affect brain rhythms could modulate thalamocortical oscillations by targeting the thalamus, sharp-wave ripples by targeting the CA3 region of the hippocampus, or alpha waves by modulating 8-12 Hz rhythms that originate in the occipital lobe. In alternative embodiments, other brain rhythms or distributed neuronal pathways are targeted by transcranial ultrasound neuromodulation. For each of the targeted rhythms, transcranial ultrasound neuromodulation may be used in some embodiments to enhance the rhythms and in other embodiments to reduce the rhythms.

At the instructed time, a transcranial ultrasound neuromodulation protocol can be delivered to stimulate the targeted region of the brain in order to activate, inhibit, or modulate its activity and induce an altered subjective experience or cognitive state for the user. Specific embodiments of neuromodulation are described herein and may include stimulation targeting primary sensory cortex, primary and secondary motor cortex, association cortex (including areas involved in emotion, executive control, language, and memory), neuromodulatory pathways, the amygdala, the hippocampal formation, and other brain regions. The transcranial ultrasound neuromodulation protocol may affect one or more of the attentional state, emotional state, or cognitive state of the recipient. Alternatively, the transcranial ultrasound neuromodulation protocol may cause one or more of the following effects: the user may be induced to consciously or unconsciously perform an act; the user may experience a state of physiological arousal or somnolence; the user may perceive a sensory stimulus or become blinded to a sensory stimulus.

An appropriate ultrasound stimulation protocol must be delivered in order to induce changes in the brain via transcranial ultrasound neuromodulation. The temporal pattern of ultrasound vibration delivered to the brain affects the induced neuromodulation. The temporal pattern of ultrasound waveforms may also affect the nature of the induced neuromodulatory effect such as neuromodulation (which may be mediated by a change in the excitability of neuronal circuits), stimulation of neuronal activity, inhibition of neuronal activity, modulation of long-term plasticity, effects on neurons and glial cells in the nervous system, or modulation of one or a plurality of the following biophysical or biochemical processes: (i) ion channel activity, (ii) ion transporter activity, (iii) secretion of signaling molecules, (iv) proliferation of the cells, (v) differentiation of the cells, (vi) protein transcription of cells, (vii) protein translation of cells, (viii) protein phosphorylation of the cells, or (ix) protein structures in the cells. In some embodiments, transcranial ultrasound neuromodulation may induce different effects concurrently in different brain regions. In some embodiments, transcranial ultrasound neuromodulation may induce effects in non-targeted brain regions. For instance, by targeting white matter tracts that project to or from a region of interest, an effect may be mediated by downstream structures (or upstream structures if signals are transmitted antidromically) that were not directly targeted by the transcranial ultrasound neuromodulation protocol.

Systems, devices, and methods described herein may incorporate one or more transcranial ultrasound neuromodulation and aTCD systems as described herein as well as components and systems for controlling their integrated operation.

FIG. 2B shows a schematic description of a method 200 to adjust targeting of transcranial ultrasound neuromodulation based on aTCD in accordance with many embodiments. Two important features may be (1) the generation of a 3-dimensional map of brain blood vessels by aTCD, and (2) transcranial ultrasound neuromodulation targeting to one or more brain regions of interest by computing the neuroanatomical location of the one or more brain regions relative to the 3-dimensional map of brain blood vessels using trigonometric or other analytical techniques.

An estimate of targeting for transcranial ultrasound neuromodulation is determined in a step 201, and the focal point of the transcranial ultrasound neuromodulation waveform is adjusted if necessary in a step 202. The transcranial ultrasound neuromodulation protocol and targeting are transmitted to device components for transcranial ultrasound neuromodulation in a step 203 and the transcranial ultrasound neuromodulation waveform is delivered to the subject in a step 207 via one or mote ultrasound transducers 204. One or more components for aTCD 213 are also used to form a 3-dimensional map of brain blood vessels in a step 208 in order to determine the relative focal point of the transcranial ultrasound neuromodulation waveform delivered to the subject. Efficacy of transcranial ultrasound neuromodulation can be assessed by one or more of: measurement of brain activity, cognitive function, or other aspect of brain function such as attention in a step 209; measurement of non-neuronal physiology such as blood pressure, heart rate, galvanic skin response, or muscle activity in a step 210; measurement of skull transmission of ultrasound in a step 211; and measurement related to the safety of transcranial ultrasound neuromodulation, including tissue heating in a step 212. The efficacy and actual targeting of transcranial ultrasound neuromodulation are compared to baseline, a desired value, and/or a value previously measured in a step 206 to determine whether targeting of transcranial ultrasound neuromodulation should be adjusted in the step 201.

Although the above steps show method 200 in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as if beneficial to the treatment.

In some embodiments, a closed loop design is incorporated to intermittently or continuously adjust transcranial ultrasound neuromodulation targeting as necessary when the aTCD system identifies a relative movement of one or more transcranial ultrasound neuromodulation transducers and a brain region target.

In order for the aTCD and transcranial ultrasound neuromodulation components of the device to functionally interact, the relative position and orientation of the one or more ultrasound transducers or transducer arrays used for each of transcranial ultrasound neuromodulation and aTCD must be either fixed or known and measurable. One additional method for tracking absolute and relative locations of the foci of each of transcranial ultrasound neuromodulation and aTCD is by tracking the small but measureable focal displacements of brain tissue caused by each, which can be monitored by ultrasound or by MRI.

In various embodiments of the invention, analytical or mathematical techniques are used for computing the location of the one or more targeted brain regions relative to a 3-dimensional blood vessel map. For instance, trigonometric functions and other appropriate mathematical techniques known in the art can be used to identify the correct brain region target in 3-dimensional space relative to a map of blood vessels.

In some embodiments of the invention, the relative position of the one or more targeted brain regions to brain blood vessels is pre-determined by computed tomography, magnetic resonance imaging, functional magnetic resonance imaging, positron emission tomography, or another form of non-invasive brain imaging that provides data concerning both the location of blood vessels and associated neuroanatomy (and/or functional neuroanatomy).

In alternative embodiments, the relative position of the one or more targeted brain regions is determined as part of a combined transcranial ultrasound neuromodulation and aTCD session. A 3-dimensional blood vessel map can be determined by aTCD and one or more assessments about the efficacy of a transcranial ultrasound neuromodulation protocol for an intended neuromodulatory effect is made relative to locations defined within this three-dimensional map. The targeting of neuromodulation can be improved or optimized based on the results of the one or more assessments of transcranial ultrasound neuromodulation efficacy, including by fTPI imaging, another functional assessment of neuronal activity, a self-report of an altered state by the subject, or cognitive assessment. The aTCD data acquired concurrently or near in time within about 5 minutes to the transcranial ultrasound neuromodulation stimulation can be used to define a target location fixed relative to the three-dimensional map of brain blood vessels generated by aTCD. The one or more measurements of brain activity, physiology, cognitive function, or other changes in the brain or body induced by transcranial ultrasound neuromodulation may include one or more of: (1) brain activity measured by one or more techniques chosen from the group of: electroencephalography (EEG), magnetoencephalography (MEG), functional magnetic resonance imaging (fMRI), functional near-infrared spectroscopy (fNIRS), positron emission tomography (PET), single-photon emission computed tomography (SPECT), computed tomography (CT), functional tissue pulsatility imaging (fTPI), or other techniques for measuring brain activity known to one skilled in the art; (2) physiology measured by one or more techniques chosen from the group of: electromyogram (EMG), galvanic skin response (GSR), heart rate, blood pressure, respiration rate, pulse oximetry, pupil dilation, eye movement, gaze direction, or another physiological measurement for instance those that can be decoded to determine a cognitive state, sleep state, physiological state, or thought, sensory perception, emotion, concept, or state of physiological arousal, sexual arousal, or attention; or (3) cognitive function assessed by one or more testing techniques chosen from the group of: a test of motor control, a test of cognitive state, a test of cognitive ability, a sensory processing task, an event related potential assessment, a reaction time task, a motor coordination task, a language assessment, a test of attention, a test of emotional state, a standardized cognitive task, or a customized cognitive task.

In some embodiments, transcranial ultrasound neuromodulation targeting is adjusted every time an aTCD protocol is performed. In alternative embodiments, transcranial ultrasound neuromodulation targeting is adjusted when an aTCD protocol determines that the position or orientation of one or more transcranial ultrasound neuromodulation components of the device have changed by an amount that exceeds a pre-determined threshold. In various embodiments, changes in transcranial ultrasound neuromodulation targeting can be assessed based on one or more of: (1) the position on the head where one or more transcranial ultrasound neuromodulation components are functionally affixed, (2) the 3-dimensional orientation of one or more transcranial ultrasound neuromodulation components, (3) changes in blood flow induced by transcranial ultrasound neuromodulation that are detected by aTCD of one or more brain blood vessels (e.g., by fTPI), (4) one or more measurements of brain activity, physiology, cognitive function, or other changes in the brain or body induced by transcranial ultrasound, (5) one or more safety assessments of transcranial ultrasound neuromodulation, (6) one or more assessments of the transmission of ultrasound waves transmitted for transcranial ultrasound neuromodulation, or (7) another assessment useful for evaluating the efficacy, safety, transmission, or targeting of transcranial ultrasound neuromodulation.

In embodiments wherein changes in transcranial ultrasound neuromodulation targeting are determined by the position on the head where one or more transcranial ultrasound neuromodulation components are functionally affixed, one or more methods and systems for determining the position of the one or more transcranial ultrasound neuromodulation components can be used. A non-exhaustive list of methods and systems for determining the position of the one or more transcranial ultrasound neuromodulation components includes: (1) systems that employ one or more cameras or video recording systems and machine vision algorithms to determine the position of the one or more transcranial ultrasound neuromodulation components; (2) systems that employ accelerometers or gyroscopes to detect movements; (3) systems that use ultrasound transducers to detect changes in signal generated by the transcranial ultrasound neuromodulation and/or aTCD transducers; or (4) other systems known for determining the position of the one or more transcranial ultrasound neuromodulation transducers.

In some embodiments, a first, detailed three-dimensional map of brain blood vessels is generated, and a second, less detailed three-dimensional map of a subset of the brain blood vessels imaged to form the detailed three-dimensional map are used to form a map of fiduciary landmarks. In some embodiments, the detailed three-dimensional map is generated intermittently, and a less detailed three-dimensional map of a subset of the brain blood vessels imaged to form the detailed three-dimensional map are used to form a map of fiduciary landmarks.

In some embodiments, a three-dimensional map of blood brain vessels is stored in a machine-readable database. The three-dimensional map may be a detailed map or a map of a subset of the imaged brain blood vessels. The database may be a component of the device wearably attached to the user. In alternative embodiments, the three-dimensional map is stored in a remote server or storage medium. Machine-readable files that represent the three-dimensional map may be transmitted wirelessly or via the Internet to the remote server. In alternative embodiments, removable storage media is used to store the three-dimensional map files and the user or another individual removes the storage media and uploads the one or more files to a computer or remote server via the Internet.

In some embodiments, aTCD is used to position and orient one or more transcranial ultrasound neuromodulation transducers in order to achieve a particular level of accuracy for targeting one or more brain regions for neuromodulation. In various embodiments, the accuracy of transcranial ultrasound neuromodulation targeting is a volume less than about 10 cm³, a volume less than about 5 cm³, a volume less than about 4 cm³, a volume less than about 3 cm³, a volume less than about 2 cm³, a volume less than about 1 cm³, a volume less than about 5 mm³, a volume less than about 4 mm³, a volume less than about 3 mm³, a volume less than about 2 mm³, or a volume less than about 1 mm³.

In embodiments wherein changes in transcranial ultrasound neuromodulation targeting are determined by changes in blood flow induced by transcranial ultrasound neuromodulation that are detected by aTCD of one or more brain blood vessels, an aTCD assessment targeting one or more blood vessels of interest near or in a brain region targeted by transcranial ultrasound neuromodulation is triggered to occur after a transcranial ultrasound neuromodulation stimulation protocol is transmitted. In some embodiments, the change in blood flow in a blood vessel of interest as assessed by aTCD is averaged across multiple transcranial ultrasound neuromodulation stimulation protocols and aTCD assessments occurring immediately following each transcranial ultrasound neuromodulation stimulation protocol. This may be different from fTPI, which gives a spatially integrated measure of bulk blood flow, primarily at the level of the capillary bed and secondary arteries and veins.

In embodiments wherein changes in transcranial ultrasound neuromodulation targeting are determined by one or more safety assessments of transcranial ultrasound neuromodulation, the safety assessment measures the thermal effects of transcranial ultrasound neuromodulation. Temperature measurements can be made by one or more techniques including by use of a thermistor, thermometer, camera-based system (e.g. an infrared camera), or other technique. In various embodiments, temperature measurements can be made of one or more of: coupling gel or other physical system for coupling ultrasound into the body; ultrasound transducer; other components of the ultrasound system; or hair, skin, skull, dura, or brain. Increased temperature in the brain is known to affect the function of neurons and neural circuits—and thus may affect cognitive state and/or cognitive function. In some embodiments, thermal effects of transcranial ultrasound neuromodulation are assessed indirectly by making one or more measurements of brain activity, physiology, cognitive state, or cognitive function.

In some embodiments wherein changes in transcranial ultrasound neuromodulation targeting are assessed by measuring skull transmission of the transcranial ultrasound neuromodulation waveform, one or more ultrasound transducers are used to detect the signature of reflected ultrasound as is done commonly in ultrasound imaging. This can be accomplished by using a pulse-echo method using ultrasound transducers with a dominant acoustic frequency of more than about 1 MHz. By measuring the relative power of reflected ultrasound with different transcranial ultrasound neuromodulation waveforms, the amount of ultrasound energy absorbed, reflected, or scattered by the skull can be determined. Ultrasound energy reflected by the skull or other part of the head or brain will return to the transducer for measurement more quickly than ultrasound energy reflected by other structural features in the brain. The timing of the expected reflected ultrasound waves can be calculated using techniques from diagnostic ultrasound imaging that are well-known to those skilled in the art of ultrasound imaging. In this embodiment, transcranial ultrasound neuromodulation waveforms for which less ultrasound energy is measured by the transducer may be more effective for neuromodulation because more energy is being transmitted through the skull.

In another embodiment wherein changes in transcranial ultrasound neuromodulation targeting are assessed by measuring skull transmission of the transcranial ultrasound neuromodulation waveform, the amount of ultrasound energy transmitted through the skull is measured by one or a plurality of transducers on the opposite side of the skull from the one or plurality of ultrasound transducers used for generating the transcranial ultrasound neuromodulation waveform. In this embodiment, the transducers used for measuring ultrasound on the contralateral side of the skull measure the amount of ultrasound energy transmitted through the skull. In this embodiment, transcranial ultrasound neuromodulation waveforms for which more ultrasound energy is measured by the one or plurality of transducers are more effective for neuromodulation because more energy is being transmitted through the skull.

In another embodiment wherein changes in transcranial ultrasound neuromodulation targeting are assessed by measuring skull transmission of the transcranial ultrasound neuromodulation waveform, one or a plurality of methods for measuring acoustic energy that do not include an ultrasound transducer such as by using a fiber optic hydrophone, photoacoustic imaging or another method for measuring acoustic energy known to one skilled in the art are used to quantify the amount of ultrasound energy transmitted through the skull, skin, dura, and brain tissue or reflected by the skull, skin, dura, and brain tissue. In this embodiment, a similar method is used as that discussed above for estimating the amount of ultrasound energy that reaches the targeted region of the brain.

In some embodiments, the system and process of adjusting the targeting of transcranial ultrasound neuromodulation based on a brain blood vessel map generated by aTCD is completed exactly once for initial targeting of transcranial ultrasound neuromodulation. In alternative embodiments, the system and process of adjusting the targeting of transcranial ultrasound neuromodulation based on a brain blood vessel map generated by aTCD is repeated in order to maintain appropriate targeting of transcranial ultrasound neuromodulation despite relative movements of the user and one or more transcranial ultrasound neuromodulation components. In various embodiments, adjusting the targeting of transcranial ultrasound neuromodulation based on a brain blood vessel map generated by aTCD is repeated about more than once, about more than about more than 5 times, about more than 10 times, about more than 15 times, about more than 20 times, about more than 25 times, about more than 30 times, about more than 35 times, about more than 40 times, about more than 45 times, about more than 50 times, about more than 75 times, about more than 100 times, about more than 200 times, about more than 250 times, about more than 300 times, about more than 400 times, about more than 500 times, about more than 1000 times, or about more than 10000 times.

Embodiments in which the adjustment of transcranial ultrasound neuromodulation targeting based on aTCD data occurs quickly may be beneficial. In alternative embodiments, the adjustment in transcranial ultrasound neuromodulation targeting may occur less than about 1 microsecond after aTCD data has been acquired and processed, less than about 1 millisecond after aTCD data has been acquired and processed, less than about 10 milliseconds after aTCD data has been acquired and processed, less than about 25 milliseconds after aTCD data has been acquired and processed, less than about 50 milliseconds after aTCD data has been acquired and processed, less than about 100 milliseconds after aTCD data has been acquired and processed, less than about 500 milliseconds after aTCD data has been acquired and processed, less than about 1 seconds after aTCD data has been acquired and processed, less than about 5 seconds after aTCD data has been acquired and processed, less than about 10 seconds after aTCD data has been acquired and processed, less than about 30 seconds after aTCD data has been acquired and processed, less than about 60 seconds after aTCD data has been acquired and processed, less than about 5 minutes after aTCD data has been acquired and processed, or at a longer time relative to the time when aTCD data has been acquired and processed.

Embodiments in which the adjustment of transcranial ultrasound neuromodulation targeting based on aTCD data occurs continuously may be beneficial. In alternative advantageous embodiments, the adjustment of transcranial ultrasound neuromodulation targeting based on aTCD data occurs intermittently. In various embodiments, transcranial ultrasound neuromodulation targeting is adjusted about every other time aTCD data is updated, about every 3 times aTCD data is updated, about every 5 times aTCD data is updated, about every 10 times aTCD data is updated, about every 20 times aTCD data is updated, about every 30 times aTCD data is updated, about every 40 times aTCD data is updated, about every 50 times aTCD data is updated, about every 100 times aTCD data is updated, about every 1,000 times aTCD data is updated, about every 10,000 times aTCD data is updated, about every 100,000 times aTCD data is updated, or less frequently. In some embodiments, transcranial ultrasound neuromodulation targeting adjustment occurs at variable times relative to aTCD data updates. Variable intervals between transcranial ultrasound neuromodulation targeting adjustments may be random, pseudo-random, or structured according to another irregular pattern relative to aTCD data updates.

In some embodiments, a 3-dimensional map of brain blood vessels generated by aTCD is stored in a machine-readable format in a database. Storing the 3-dimensional map for later access can permit transcranial ultrasound neuromodulation targeting to a particular brain region during a subsequent transcranial ultrasound neuromodulation and aTCD session. In various embodiments, the database includes additional information specific to one or more of the device components, device operation, user, and one or more targeted brain regions.

In some embodiments, 3-dimensional map of brain blood vessels generated by aTCD that has been previously stored in a machine-readable format in a database is accessed in order to define a baseline brain blood vessel map.

An aTCD and/or transcranial ultrasound neuromodulation ultrasound protocol may be initiated following positioning, orientation and adjustment of a framework structure, probe mount, and ultrasound probe. In some embodiments, an associated aTCD ultrasound monitoring system having a display is operated to identify and locate a probe illumination area, the user or another operator manipulates the ultrasound probe and/or probe mount to match the probe illumination area with a target marked on the display, and the user or operator then locks the probe and/or probe mount into place. The ultrasound monitoring system may be programmed to alert the user, or an operator, if the probe illumination area strays from the target, or if or when the probe needs to be repositioned and the target re-acquired. Various types of protocols for automated target location and station-keeping may be implemented.

The user data recording and storage device may be operated to collect and/or store data continuously or intermittently and may optionally have analytical and/or display capabilities as well. In some embodiments, manual activation and shut-off mechanisms are provided, enabling a subject to activate and inactivate the data acquisition devices and record and store data. Data acquisition routines may involve, for example, acquiring data from one or more data acquisition devices at certain time intervals or during certain physiological states, acquiring data for certain time intervals, and transmitting and storing the data in specified databases or in one or more storage location(s).

Ambulatory devices may be provided with individual identifiers and may have data transmit-receive capabilities that enable acquired data to be transmitted to a remote data storage and/or analysis system, and that enable control systems, data acquisition and analysis routines, limits, and the like to be transmitted from a remote location to the ambulatory device. Data may be transferred from an ambulatory device.

Ambulatory devices may also have localization capabilities incorporating VHF, GPS, satellite and/or triangulation location systems. These systems are capable of notifying care-givers or services having a companion receiver, in real time, of anomalies in a subject's physiology, location or the like, thus allowing the monitoring entity to take action to find and assist the subject.

Vibroacoustography stimulation makes use of confocal transducers run at separate frequencies f₁ and f₂, typically chosen to be in the range between about 1 MHz and about 5 MHz due to the regular use of ultrasound systems operating at these acoustic frequencies for medical imaging applications. The dominant frequency at the intersection of the two beams is equal to the difference of their frequencies due to constructive and destructive interference. At the focus, the high-frequency ultrasound creates tissue movement with a frequency equal to f₂−f₁. In many embodiments, emissions from the vibrating tissue at the difference frequency can be monitored. In many embodiments as described herein, vibroacoustography stimulation is used to generate focal tissue movement with a dominant frequency of, for instance, about 350 kHz to modulate brain function.

In many embodiments, the two or more high ultrasound frequencies are sufficiently coherent so as to interfere and produce one or more beat frequencies at the target site.

In many embodiments, the vibroacoustic stimulation based on the difference between two or more interfering high frequencies of ultrasound as described herein, produces an unexpected result in that the confocal high ultrasound frequencies (e.g. above 1 MHz) can provide stimulation with amounts of energy similar to lower ultrasound frequencies (e.g. below 1 MHz). Although reference is made to vibroacoustography to stimulate neuronal tissue by way of explanation in accordance with embodiments, a person of ordinary skill in the art will recognize that the vibroacoustic stimulation as described herein produces an unexpected result in that the modulation of two or more high frequencies can stimulate tissue with lower amounts of energy than either of the high frequencies or an average of the high frequencies.

In many embodiments, the beat frequency (f_{beat}) corresponds to the difference between the second frequency (f₂) and the first frequency (f₂), as expressed with the equation:

f_{beat}=f₂−f₁.

In many embodiments, the average frequency (f_{avg}) corresponds to the average of the second frequency (f₂) and the first frequency (f₂), as expressed with the equation:

f_{avg}(f₂+f₁)/2.

The waveform resulting from the combination of two or more interfering high frequencies corresponds to the modulation of the average frequency with the beat frequency can be expressed with the equation:

sin(2πf₁t)+sin(2πf₂t)=2 cos(2πf_{beat}t)sin(2πf_{avg}t).

The modulation of the average frequency with the beat frequency, produces an unexpected result in that the amount of energy required to stimulate tissue is substantially lower than for any of the first high frequency, the second high frequency, or a high frequency within the range of frequencies between the first high frequency and the second high frequency, for example the average frequency f_{avg}.

In many embodiments, results similar to the beat frequency provided with the confocal array can be provided with a waveform similar to the beat frequency waveform and can provide similar results in accordance with embodiments described herein. In many embodiments, an array of ultrasound transducers can be configured to emit a focused ultrasound beam having a frequency corresponding to average frequency (f_{avg}) of two high ultrasound frequencies as described herein, and the focused beam having the frequency similar to the average frequency can be modulated with a frequency similar to the beat frequency. In specific embodiments, the frequency corresponding to the average frequency may comprise a frequency of 1.10 MHz, and the frequency corresponding to the beat frequency may comprise 0.2 MHz, for example, and the 1.10 MHz focused ultrasound can be amplitude modulated at 0.2 MHz, so as to produce a waveform similar to a first high ultrasound frequency of 1.0 MHz and a second high ultrasound frequency of 1.2 MHz producing a beat frequency of 0.2 MHz and an average frequency of 1.1 MHz, for example. This amplitude modulation of a high ultrasound frequency carrier wave (e.g. above 1 MHz) with a lower frequency modulation signal (e.g. less than 1 MHz) can stimulate tissue with lower amounts of energy than the high frequency component, and can provide an unexpected result in that the amounts of energy capable of stimulation with the high frequency amplitude modulated ultrasound signal are decreased substantially as compared with the high frequency without amplitude modulation. In many amplitude modulated embodiments, the modulation signal of the amplitude modulated ultrasound beam is present throughout the entire beam. In at least some embodiments, it may be desirable to provide decreased interaction of the treatment beam with tissue away from the treatment site.

In many embodiments, the overlap of the two or more high frequency ultrasound beams at the target site increases interaction of the beams at the treatment site and decreases interaction of the treatment beams away from the target site where the beams do not overlap substantially, in order to provide more accurate modulation of neuronal activity at the target site. In these embodiments, vibroacoustic stimulation induces a beat frequency modulation where the two confocal beams overlap substantially as a result of interference of two or more high frequency ultrasound beams, for example at the target site. The two or more confocal overlapping beams at the target site that do not overlap substantially away from the target site may be appropriate in embodiments where it is desirable to limit stimulation to the target site and to inhibit stimulation away from the target site, and the non-overlapping beams away from the target site can provide this decreased tissue interaction.

It is contemplated, that in some combinational embodiments, the overlapping beams may be amplitude modulated, for example when combined with mapping as described herein.

Although the experimental section as described herein makes reference to studies with mice, mice are a mammal comprising a cranium, motor cortex and brain making the mouse a suitable experimental model for human and non-human subjects in accordance with embodiments as described herein. A person of ordinary skill in the art can construct apparatus and perform methods suitable for use on human subjects and non-human subjects in accordance with embodiments as described herein. Further, stimulation of the movement cortex is provided merely by way of example due to ease of measurement of efficacy, and similar results can be obtained with one or more many neural targets sites, such as one or more of the primary sensory cortex, primary and secondary motor cortex, association cortex (including areas involved in emotion, executive control, language, and memory), other regions of the cerebral cortex, the limbic system (including the amygdale), hippocampus, parahippocampal formation, entorhinal cortex, subiculum, thalamus, hypothalamus, white matter tracts, brainstem nuclei, cerebellum, neuromodulatory system, or other brain regions.

Experimental Section

Here, the inventors discuss experimental results of neuromodulation by transcranial vibroacoustography stimulation in lightly anesthetized mice with high anatomical specificity in accordance with embodiments described herein. Specifically, the inventors have assessed movements induced by vibroacoustography stimulation.

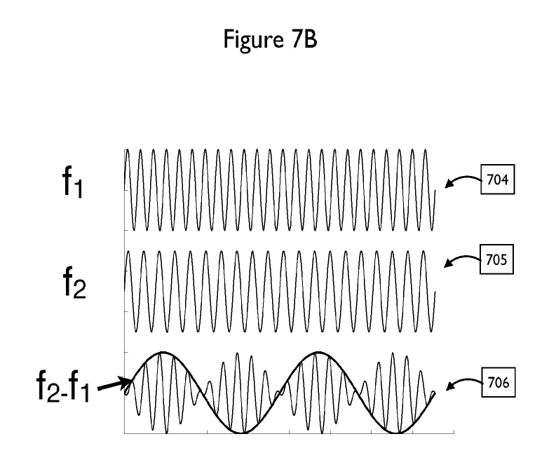

FIG. 7A shows an experimental setup for delivering and assessing ultrasound waveforms for vibroacoustography stimulation, in accordance with embodiments as described herein. The resulting acoustic waveforms can be assessed in water tank 701 containing tissue sample containing a target tissue 702 targeted with two annular ultrasound arrays 703 delivering acoustic energy with a first dominant frequency 704 of f₁ and a second dominant frequency 705 of f₂, respectively, to a common focal point within the target site comprising target tissue 702. Hydrophone 707 can measure the resulting waveform that occurs due to interference 706 which is equal to (f₂−f₁). The signal from the hydrophone can be amplified, filtered by hardware 708 and then displayed on oscilloscope 709 timed to external trigger 710 that can also trigger ultrasound delivery. FIG. 7B shows a schematic of higher acoustic frequency waveforms f₁ 704 and f₂ 705 and a dominant slower frequency caused by interference of these two waveforms at the target tissue site 706.

The plurality of ultrasound arrays 703 comprises a first ultrasound array and a second ultrasound array. The first ultrasound array is configured to provide a first focused ultrasound beam comprising first one or more high frequencies such as first dominant frequency 704, and the second ultrasound array is configured to provide a second focused ultrasound beam comprising the second one or more high frequencies such as second dominant frequency 705. The first beam is focused to the common focal point within target site with the second beam is focused to the common focal point such that the first beam and the second beam comprise the confocal arrangement. The first array and the second array are shown configured with a first annular array and a second annular array. The first array and the second array are arranged such that the first array does not overlap substantially with the second array, and such that the first beam and the second beam do not overlap substantially away from the target site, in order to decrease interaction of the beams with tissue away from the target site. As the beams converge toward the target site comprising target tissue 702, the first beam overlaps at least partially with the second beam in order to provide increased interaction of the beams at the target site. The first array and the second array can be arranged such that the beams substantially overlap only within a target site of the brain, for example. The substantial overlap may comprise an overlap of at least 50% of the area of the full width half maximum of each beam.

The ultrasound device can be, for example, a device made by Sonic Concepts, WA and has been modified in accordance with the embodiments as described herein. The device may comprise a confocal, dual-annular array with a focal length of 65 mm and a roughly cylindrical focal volume (at the half-pressure maximum) measuring approximately 8 mm in the axial direction and 1 mm in the radial direction. Each annulus delivered bursts of ultrasound, described below, one with a carrier frequency of 2.25 MHz and the other of 1.75 MHz. Each stimulation protocol, delivered at 1 Hz over ten seconds, comprised 88 pulses of ultrasound emitted with a pulse repetition frequency of 1.5 kHz, each lasting for 200 microseconds. Taken together, these transducers emitted a spatial peak, temporal average intensity of 8 W/cm², as measured with a needle hydrophone in a tank of degassed water.

To facilitate transmission of this ultrasound from the transducer into the brains of the test mice, a plastic cone was placed over the device, into which degassed water was placed. For effective coupling to the mouse's head, hair was removed by first shaving, then applying Nair™ to the top of the mouse's head. The open, distal end of the cone was covered with acoustically transparent soft latex and the proximal end was acoustically coupled to the mouse skull with ultrasound gel. A micro-positioning system was used to move the ultrasound device over the head of the mouse. The targeting of the ultrasound device was determined with a laser-positioning system that identified the projection of the focus on the top of the mouse's head. The vertical positioning of the annular rings was chosen so that the foci of the annular ultrasound rings were in the brain of the mouse.

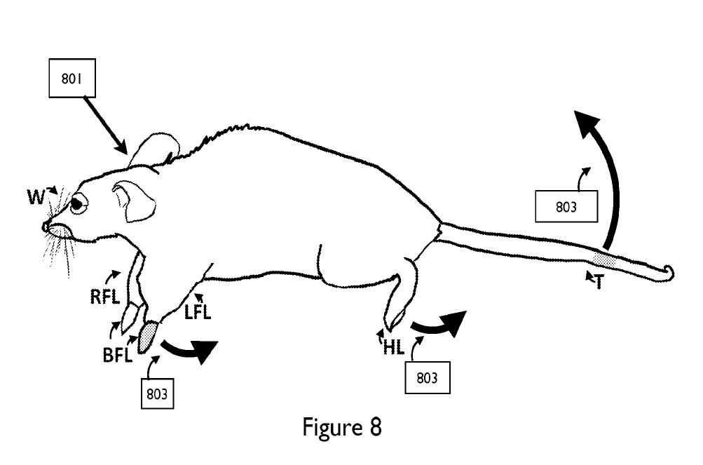

FIG. 8 shows schematic of test mouse 801 indicating the categories of movements observed in response to vibroacoustographic stimuli as described herein. FIG. 8 includes various descriptors: RFL represents the right front leg, LFL represents left front leg, BFL represents both front legs, HL represents hind legs, W=whiskers, and T represents the tail. Arrows 803 indicate the type of movements observed.

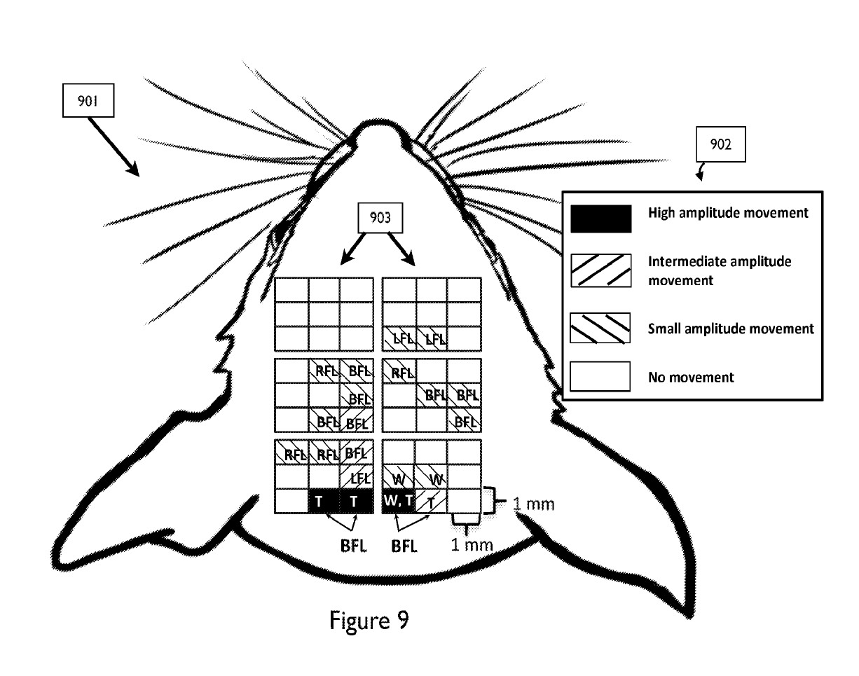

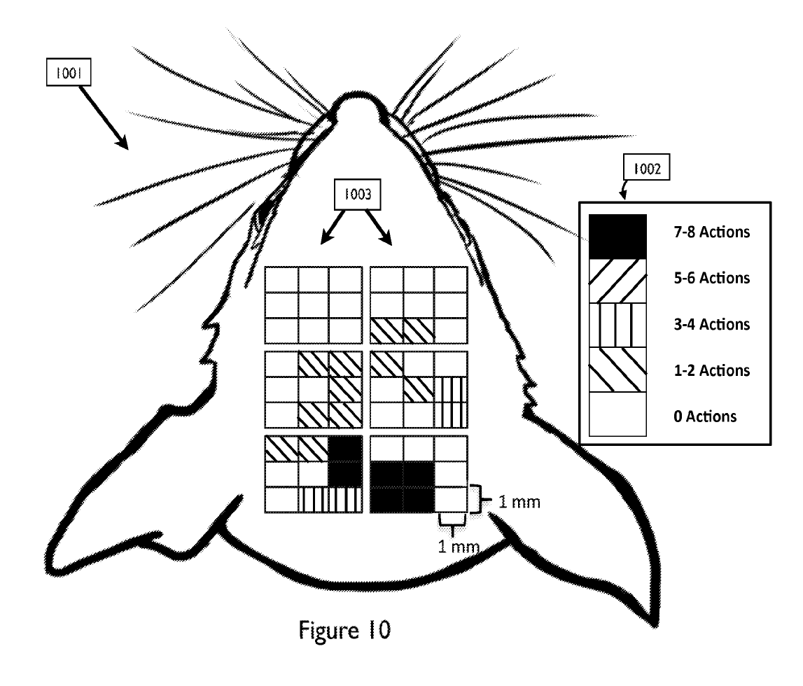

FIG. 9 and FIG. 10 show schematics of the top of mouse's head 901, 1001. To comprehensively assess motor responses, we defined a grid of 81 boxes 902, 1002 measuring approximately 1 mm to a side. Vibroacoustographic stimuli were delivered transcranially at the location of each position in the grid and observed the resulting motor responses of the mice. In FIG. 9, the background in each grid location indicates the intensity of movement observed according to legend 902, as well as the one or more types of movement elicited according to the abbreviations listed in legend 802. In FIG. 10, the background in each grid location indicates the number of movements (of any type) observed during a 10 second stimulation according to legend 1002. Most movement was induced when the ultrasound was applied into the mouse's right posterior portion of brain.

The results presented for this mouse are representative of experimental observations in other test subjects: focal induction of motion of different kinds, where movement of the neuromodulatory probe by even just 1 mm significantly changed both the type and amplitude of movement.

As a control experiment, ultrasound was beamed into the brains of other mice with the same parameters as above, but this time with each annular transducer transmitting ultrasound with the same frequency (2 MHz) rather than with slightly different frequencies as above. In this way a difference frequency of 500 kHz as above was not induced: the ultrasound protocol consisted of a single frequency, again of 2 MHz. In general, while this control ultrasound did occasionally induce observable motion, such motion occurred at substantially fewer places within the 81-place grid, occurred substantially less often as a percentage of a given 10-second application of ultrasound, and the associated movements were much more subtle than those observed when using two different ultrasound frequencies according to the vibroacoustography stimulation protocol described above. Therefore, these experimental results show increased stimulation with similar amounts of energy.

In some embodiments, functional tissue pulsatility imaging (fTPI) is used to measure brain activity noninvasively through the use of transcranial ultrasound. fTPI may be an advantageous technique for functionally mapping brain regions to be targeted by transcranial ultrasound neuromodulation on a functional basis and to serve as a functional fiduciary landmark relative to aTCD maps of brain blood vessels. In these embodiments, targeting of transcranial ultrasound neuromodulation can be informed by both relative position information derived from aTCD signals and functional assessments of neuronal activity generated by fTPI protocols. For example, combined fTPI, aTCD, and transcranial ultrasound neuromodulation can be used to ‘chase’ patterns of neuronal activity, such as chasing abnormal activity occurring due to seizure.

Definitions

In this application, the terms “subject”, “user”, and “recipient” are used interchangeably.

In this application, the terms ‘brain stimulation’, ‘neuromodulation’, and ‘neuronal activation’ are used interchangeably to refer to invasive or non-invasive techniques to alter the excitability, action potential rate, vesicular release rate, or other biochemical pathway in neurons or other cell types in the brain, in accordance with embodiments as described herein.

In this application, the terms “transcranial ultrasound neuromodulation”, “transcranial ultrasound neuromodulation protocol”, ‘transcranial ultrasound neuromodulation stimulation protocol’, ‘transcranial ultrasound neuromodulation stimulation waveform’, ‘ultrasound stimulation protocol’, ‘ultrasound stimulation waveform,” and “transcranial ultrasound neuromodulation stimulation” are used interchangeably, in accordance with embodiments as described herein.

In this application, the term “pulse length” is defined as the amount of time of a non-interrupted tone burst of one or more ultrasound acoustic wave frequency components, in accordance with embodiments as described herein.

In this application, the term “pulse repetition period” is defined to be the amount of time between the onset of consecutive ultrasound pulses, in accordance with embodiments as described herein. The “pulse repetition frequency” is equivalent to the inverse of the “pulse repetition period”, in accordance with embodiments as described herein.

In this application, the term “transcranial ultrasound neuromodulation waveform” is defined to be ultrasound delivered with a pulsed or continuous wave construction or more complex waveform, delivered over a period of time, in accordance with embodiments as described herein. Transcranial ultrasound neuromodulation waveforms may include a specified number of pulses that may be repeated at the pulse repetition frequency, in accordance with embodiments as described herein. In some embodiments, a transcranial ultrasound neuromodulation waveform is composed of a single continuous wave tone burst of greater than about one second that is not repeated. In such embodiments, the “pulse length” and “transcranial ultrasound neuromodulation waveform duration” may be about equal.

In this application, the term “transcranial ultrasound neuromodulation waveform component” may refer to be a feature of a transcranial ultrasound neuromodulation waveform that, in isolation, is insufficient to fully define a transcranial ultrasound neuromodulation waveform, in accordance with embodiments as described herein.

In this application, the term “transcranial ultrasound neuromodulation repetition period” is defined to be the amount of time of between the onset of consecutive transcranial ultrasound neuromodulation waveforms, in accordance with embodiments as described herein. The “transcranial ultrasound neuromodulation repetition frequency” may correspond to the inverse of the “transcranial ultrasound neuromodulation repetition period”, in accordance with embodiments as described herein.

In this application, the term “transcranial ultrasound neuromodulation assessment” is used to refer to one more measurements that assess one or more of the safety, efficacy, or efficiency of ultrasound transmission to the one or more targeted brain regions, in accordance with embodiments as described herein.

In this application, the terms “vibroacoustography”, “vibroacoustography stimulation” and “vibroacoustographic stimuli” are used to refer to a transcranial neuromodulation in which confocal ultrasound waves differing in acoustic frequency by a frequency effective for transcranial ultrasound neuromodulation interfere to generate vibrational forces effective for transcranial ultrasound neuromodulation, in accordance with embodiments as described herein.

In this application, the term “acoustic contrast agent” is used to refer to a substance that typically consist of stable bubbles and are filled with compressible fluids or, more typically, gas, coated with a variety of materials to help facilitate their stabilization after they are injected into the blood stream, in accordance with embodiments as described herein.

In this application, the terms “functional tissue pulsatility imaging” and “fTPI” are used to refer to an ultrasonic technique to map brain function by measuring changes in tissue pulsatility due to changes in blood flow with neuronal activation, in accordance with embodiments as described herein.

As used in the specification and the appended claims, the singular forms “a,” “an,” and “the” include plural referents unless the context clearly dictates otherwise. Thus, for example, reference to “an ultrasound waveform” may include mixtures of two or more ultrasound waveforms, and the like, in accordance with embodiments as described herein and may be claimed accordingly.

Ranges can be expressed herein as from “about” one particular value, and/or to “about” another particular value in accordance with embodiments as described herein. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed, and may be claimed accordingly. It is also understood that the throughout the application, data is provided in a number of different formats, and that this data, represents endpoints and starting points, and ranges for any combination of the data points, and may be claimed accordingly. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to encompass the following:

“Optional” or “optionally” means that the subsequently described event or circumstance may or may not occur, and that the description includes instances where said event or circumstance occurs and instances where it does not.

The term “treating” refers to one or more of inhibiting, preventing, curing, reversing, attenuating, alleviating, minimizing, suppressing or halting the deleterious effects of a disease, or causing the reduction, remission, or regression of a disease, or providing ultrasound to provide a beneficial effect in order to affect a neurological perception or sensation of subject in accordance with embodiments as described herein. Those of skill in the art will understand that various methodologies and assays can be used to assess the development of a disease, and similarly, various methodologies and assays may be used to assess the reduction, remission or regression of the disease, and to provide a desired sensation or perception of a subject.

“Increase” may be defined throughout as less than a doubling such as an increase of 5%, 10%, or 50%, for example, or as an increase of 1, 2, 3, 4, 5, 6, 7, 8, 9, 10, 11, 12, 13, 14, 15, 16, 17, 18,19, 20 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 51, 52,53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 400, or 500 times increase as compared with basal levels or a control, and it is contemplated that any of these specific values can be provided in accordance with embodiments as described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An apparatus to treat a subject with ultrasound energy, the apparatus comprising:
two or more ultrasound transducers to direct ultrasound energy transcranially to a neuronal target site of the subject;
circuitry coupled to the two or more ultrasound transducers to drive the two or more ultrasound transducers with two or more ultrasound frequencies in order to treat the subject with one or more ultrasound frequencies less than the two or more ultrasound frequencies.

2. The apparatus of claim 1, wherein the two or more transducers and the circuitry are configured to map the subject transcranially and to track a tissue site of the subject with at least one of the two or more ultrasound frequencies and to treat the target site with the two or more ultrasound frequencies.

3. The apparatus of claim 2, wherein the circuitry and transducers are configured to adjust an angle of the target site relative to the two or more ultrasound transducers in response to movement of the tracked tissue relative to the two or more ultrasound transducers.

4. The apparatus of claim 3, wherein the circuitry and transducers are configured to adjust the angle and a depth of the target site relative to the two or more ultrasound transducers in response to movement of the tracked tissue relative to an angle and a depth of the two or more ultrasound transducers.

5. The apparatus of claim 2, wherein the tracked tissue site comprises an untreated tissue site.

6. The apparatus of claim 2, wherein the tracked tissue site is the same as the target site.

7. The apparatus of claim 1, wherein the two or more transducers and the circuitry are configured to map the subject transcranially and to track a tissue site of the subject with a tracking frequency different from the two or more ultrasound frequencies for treating the subject.

8. The apparatus of claim 1, wherein the two or more transducers comprise confocal transducers to direct the ultrasound to the target site and wherein the circuitry is configured to drive the confocal transducers with the two or more ultrasound frequencies comprising a first ultrasound frequency and a second ultrasound frequency and wherein the one or more frequencies comprises a difference between the first ultrasound frequency and the second ultrasound frequency in order to vibrate the target site with an ultrasound frequency based on the difference between the first frequency and the second frequency.

9. The apparatus of claim 1, wherein the two or more ultrasound transducers and the circuitry are configured to map brain blood vessels of the subject with a first transcranial ultrasound configuration and to treat the target site with second transcranial ultrasound configuration.

10. The apparatus of claim 1, wherein the two or more ultrasound frequencies comprise frequencies within a range from about 1 MHz to about 15 MHz and wherein the one or more frequencies less than the ultrasound frequency comprise frequencies less than about 1 MHz.

11. A method of treating a subject with ultrasound energy, the method comprising:
directing ultrasound energy comprising two or more ultrasound frequencies to a target site, wherein the two or more frequencies induce vibration of the target site with one or more ultrasound frequencies less than the two or more ultrasound frequencies to modulate neuronal activity at the target site.

12. The method of claim 11, further comprising mapping the subject transcranially and tracking a tissue site with at least one of the two or more ultrasound frequencies.

13. The method of claim 12, further comprising adjusting an angle of ultrasound energy direction to the target site in response to movement of the tracked tissue site.

14. The method of claim 13, further comprising adjusting the angle and a depth of the target site in response to movement of the tracked tissue site.

15. The method of claim 12, wherein the tracked tissue site comprises an untreated tissue site.

16. The method of claim 12, wherein the tracked tissue site is the same as the target site.

17. The method of claim 11, further comprising mapping the subject transcranially and tracking a tissue site with a tracking frequency different from the two or more ultrasound frequencies to modulate neuronal activity at the target site.

18. The method of claim 11, wherein the two or more ultrasound frequencies comprise a first ultrasound frequency and a second ultrasound frequency,
wherein the one or more ultrasound frequencies less than the two or more ultrasound frequencies comprise a difference between the first ultrasound frequency and the second ultrasound frequency, and
wherein directing ultrasound energy comprises vibrating the target site with an ultrasound frequency based on the difference between the first ultrasound frequency and the second ultrasound frequency.

19. The method of claim 11, wherein directing ultrasound energy comprises mapping brain blood vessels of the subject with a first transcranial ultrasound configuration and treating the target site with a second transcranial ultrasound configuration.

20. The method of claim 11, wherein the two or more ultrasound frequencies comprise frequencies within a range from about 1 MHz to about 15 MHz and wherein the one or more frequencies less than the ultrasound frequency comprise frequencies less than about 1 MHz.

21. The apparatus for treating a subject with ultrasound energy as in any one of claims 1-7, the apparatus further comprising a processor comprising tangible medium configured to implement the method of one of claims 11 to 20.

22. A system for transcranial ultrasound neuromodulation that uses Doppler ultrasound imaging for targeting one or more brain regions.

23. The system of claim 22, wherein the transcranial Doppler imaging system is an automated transcranial Doppler (aTCD) imaging system.

24. The system of claim 23, wherein a three-dimensional map of brain blood vessels is generated by aTCD.

25. The system of claim 23, wherein
a first three-dimensional map of brain blood vessels is generated with aTCD and stored in machine-readable format;
one or more subsequent brain blood vessel maps generated by aTCD image a subset of one or more brain blood vessels mapped in the detailed three-dimensional map; and
the one or more subsequent brain blood vessel maps generated by aTCD are used to target transcranial ultrasound neuromodulation to one or more brain regions.

26. The system of claim 25, wherein the detailed three-dimensional map of brain blood vessels generated by aTCD is repeated intermittently.

27. The system of claim 23, wherein the three-dimensional map of brain blood vessels generated by aTCD serves as a fiduciary landmark for targeting transcranial ultrasound neuromodulation.

28. The system of claim 24, wherein the three-dimensional map of brain blood vessels generated by aTCD that serves as a fiduciary landmark for targeting transcranial ultrasound neuromodulation is created before a transcranial ultrasound neuromodulation session.

29. The system of claim 24, wherein the three-dimensional map of brain blood vessels generated by aTCD that serves as a fiduciary landmark for targeting transcranial ultrasound neuromodulation is updated during a transcranial ultrasound neuromodulation session.

30. The system of claim 29, wherein a change in the relative position of the one or more targeted brain regions and one or more transcranial ultrasound neuromodulation transducers during a transcranial ultrasound neuromodulation session is determined by comparing two or more aTCD images.

31. The system of claim 30, wherein the position or orientation of one or more transcranial ultrasound neuromodulation transducers is automatically changed based on the relative movement detected in order to maintain targeting of one or more brain regions.

32. The system of claim 30, wherein the focusing characteristics of one or more transcranial ultrasound neuromodulation transducers is automatically changed based on the relative movement detected in order to maintain targeting of one or more brain regions.

33. The system of claim 30, wherein the accuracy of targeting for transcranial ultrasound neuromodulation is less than 1 cm³.

34. The system of claim 30, wherein the accuracy of targeting for transcranial ultrasound neuromodulation is less than 1 mm³.

35. The system of claim 24, wherein the three-dimensional map of brain blood vessels is stored in machine readable format.

36. The system of claim 24, wherein the machine readable three-dimensional map of brain blood vessels is stored in one or more components of the device wearably attached to the subject.

37. The system of claim 36, wherein the machine readable three-dimensional map of brain blood vessels is stored remotely on a server.

38. The system of claim 29, wherein the three-dimensional map of brain blood vessels generated by aTCD is updated about more than once per hour.

39. The system of claim 29, wherein the three-dimensional map of brain blood vessels generated by aTCD is updated about more than once per minute.

40. The system of claim 29, wherein the three-dimensional map of brain blood vessels generated by aTCD is updated about more than once per second.

41. The system of claim 29, wherein the three-dimensional map of brain blood vessels generated by aTCD that serves as a fiduciary landmark for targeting transcranial ultrasound neuromodulation is updated continuously during a transcranial ultrasound neuromodulation session.

42. The system of claim 22, wherein the spatial-peak, temporal-average intensity in brain tissue for transcranial ultrasound neuromodulation is chosen from a range of about 0.0001 mW/cm2 to about 1 W/cm2.

43. The system of claim 22, wherein the heating of brain tissue at the target location is no more than about 2 degrees Celsius for no more than about 5 seconds.

44. The system of claim 22, wherein the acoustic frequency for transcranial ultrasound neuromodulation is in a range between about 100 kHz and about 1 MHz.

45. The system of claim 44, wherein the acoustic frequency is modulated during the transcranial ultrasound neuromodulation protocol.

46. The system of claim 22, wherein the acoustic frequency for aTCD is in a range between about 0.5 MHz and about 15 MHz.

47. The system of claim 46, wherein the acoustic frequency is modulated during aTCD imaging.

48. The system of claim 22, wherein two confocal ultrasound transducers differing in dominant acoustic frequency by an acoustic frequency appropriate for transcranial ultrasound neuromodulation are targeted at a site of tissue to be modulated by transcranial ultrasound neuromodulation.

49. The system of claim 22, wherein a transcranial ultrasound neuromodulation protocol is targeted to multiple brain regions with one or more ultrasound transducers.

50. The system of claim 22, wherein multiple transcranial ultrasound neuromodulation protocols differing in one or more of spatial-peak, temporal-average intensity, acoustic frequency, pulse length, pulse repetition frequency, and number of pulses are delivered concurrently or in series to one or more brain regions from one or more ultrasound transducers.

51. The system of claim 22, wherein the transcranial ultrasound neuromodulation transducers target one or more brain region chosen from the list of: primary sensory cortex, primary and secondary motor cortex, association cortex (including areas involved in emotion, executive control, language, and memory), other region of cerebral cortex, the limbic system (including the amygdala), hippocampus, parahippocampal formation, entorhinal cortex, subiculum, thalamus, hypothalamus, white matter tracts, brainstem nuclei, cerebellum, neuromodulatory system, or other brain region.

52. The system of claim 22, wherein the transcranial ultrasound neuromodulation stimulation is perceived subjectively by the recipient as a sensory perception, movement, concept, instruction, other symbolic communication, or modifies the recipient's cognitive, emotional, physiological, attentional, or other cognitive state.

53. The system of claim 22, wherein the system includes one or more components for measuring brain activity that takes the form of one or a plurality of: electroencephalography (EEG), magnetoencephalography (MEG), functional magnetic resonance imaging (fMRI), functional near-infrared spectroscopy (fNIRS), positron emission tomography (PET), single-photon emission computed tomography (SPECT), computed tomography (CT), or other techniques for measuring brain activity.

54. The system of claim 53, wherein brain activity is measured by detecting changes in hemodynamics with aTCD or fTPI.

55. The system of claim 29, wherein the system includes one or more components for a physiological measurement of the body that takes the form of one or a plurality of: electromyogram (EMG), galvanic skin response (GSR), heart rate, blood pressure, respiration rate, pulse oximetry, pupil dilation, eye movement, gaze direction, or other physiological measurement.

56. The system of claim 29, wherein the transcranial ultrasound neuromodulation protocol includes modulation of one or more stimulus parameters chosen from spatial-peak, temporal-average intensity, acoustic frequency, pulse repetition frequency, number of pulses, and pulse length.

57. The system of claim 29, wherein broadband ultrasound is generated at the site of tissue to be modulated through the use of an acoustic contrast agent.
